(19) United States
(12) Patent Application Publication
(10) Pub. No.: US 2017/0246210 A1
(43) Pub. Date: Aug. 31, 2017

(54) EX-VIVO INDUCED REGULATORY MESENCHYMAL STEM CELLS OR MYELOID-DERIVED SUPPRESSOR CELLS AS IMMUNE MODULATORS

(71) Applicant: HADASIT MEDICAL RESEARCH SERVICES & DEVELOPMENT LTD, Jerusalem (IL)

(72) Inventors: Reuven OR, Tsfon-Yehuda (IL); Liad HINDEN, Jerusalem (IL); Osnat HAZAN, Harey-Yehuda (IL)

(21) Appl. No.: 15/328,477

(22) PCT Filed: Jul. 23, 2015
(86) PCT No.: PCT/IL2015/050759

§ 371 Date: May 9, 2017

Related U.S. Application Data
(60) Provisional application No. 62/119,428, filed on Feb. 23, 2015.
(60) Provisional application No. 62/027,796, filed on Jul. 23, 2014.

(51) Int. Cl. A61K 35/28; C12N 5/0775; A61K 9/00

(52) U.S. Cl. CPC A61K 35/28; A61K 9/0019; C12N 5/0663; C12N 2501/15; C12N 2501/24

(57) ABSTRACT

A composition comprising TGFβ, an inflammatory agent and a tryptophan indoleamine-2,3 dioxygenase (IDO) metabolite, in particular, TGFβ, IFNγ and kynurenine, is provided, as well as regulatory mesenchymal stem cell lines or myeloid-derived suppressor cell lines obtained by contacting mesenchymal stem cell lines or myeloid-derived suppressor cell lines, respectively, with the composition. Methods for inhibiting proliferation of T cells, reducing Th17 and Tc17 differentiation of activated T cells and inflammation or for treating inter alia graft-versus-host disease (GVHD), comprising administering the cell lines, are further provided.

### FIELD OF THE INVENTION

The present invention relates in general to the use of regulatory mesenchymal stem or stromal cells or regulatory myeloid-derived suppressor cells for treatment of immune disorders.

### BACKGROUND OF THE INVENTION

Mesenchymal stem or stromal cells (MSCs) are multipotent progenitor cells characterized by their intrinsic self-renewal capacity and multilineage differentiation potentials. They are biologically available and can be isolated and expanded ex-vivo from many tissues throughout the body. MSCs express very low levels of major histocompatibility complex (MHC) antigens allowing them to remain low immunogenic. One of the most useful properties of MSCs is that these cells exert powerful anti-inflammatory and immune-suppressive function (Le Blanc, Tammik et al. 2003, Aggarwal and Pittenger 2005, Rasmusson, Ringden et al. 2005, Le Blanc and Mougiakakos 2012). MSCs were demonstrated to have a regulatory effect on various immune cells including T cells, B cells, dendritic cells and macrophages. MSCs inhibit T cell proliferation (Aggarwal and Pittenger 2005, Glennie, Soeiro et al. 2005), and influence T cell differentiation, inhibiting the development of Th17 and Tc17 cells (Ghannam, Pene et al. 2010, Glenn, Smith et al. 2014).

Over the past decade, the immunomodulatory functions of MSCs have triggered great interest in their application towards treating various immune disorders, including graft-versus-host disease (GVHD), transplanted organ rejection and autoimmune diseases (Ghannam, Pene et al. 2010, Liang, Zhang et al. 2010, Yamout, Hourani et al. 2010, Figueroa, Carrion et al. 2012, Wang, Qu et al. 2012, Casiraghi, Perico et al. 2013, Forbes, Sturm et al. 2014). While previous large-scale clinical trials using MSCs therapy had inconclusive results (Allison 2009, Ankrum and Karp 2010, Kim, Im et al. 2013), it is possible that these results are due to the varied cytokine environments MSCs encounter in-vivo. The immunosuppressive quality of MSCs in-vivo are only seen when they are exposed to sufficiently high levels of specific cytokines (Li, Ren et al. 2012). When not exposed to these cytokines, MSCs tend to lose their immunosuppressive qualities and promote lymphocyte proliferation (Stagg, Pommey et al. 2006, Romieu-Mourez, Francois et al. 2009).

There is therefore a need to improve the outcome of MSC immunomodulatory treatment.

### SUMMARY OF INVENTION

In one aspect, the present invention is directed to a composition comprising TGFβ, an inflammatory agent, IFNγ and a tryptophan indoleamine-2,3 dioxygenase (IDO) metabolite.

The purpose of the composition and its characteristic feature is that, upon its contact with a primary mesenchymal stem cell line or primary myeloid-derived suppressor cell line it imposes on the cell line an immunosuppressive phenotype.

In another aspect, the present invention is directed to a method for obtaining a regulatory mesenchymal stem cell line or a regulatory myeloid-derived suppressor cell line comprising contacting a primary mesenchymal stem cell line or a primary myeloid-derived suppressor cell line, respectively, ex-vivo with (i) a composition comprising TGFβ, a composition comprising an inflammatory agent and a composition comprising a tryptophan IDO metabolite; or (ii) the composition comprising a combination of TGFβ, an inflammatory agent and a tryptophan IDO metabolite as described above, thereby obtaining the regulatory mesenchymal stem cells.

In yet another aspect, the present invention provides a regulatory mesenchymal stem cell line or a regulatory myeloid-derived suppressor cell line obtained according to the methods defined herein; or a regulatory mesenchymal stem cell line or regulatory myeloid-derived suppressor cell line characterized by elevated levels of iNOS, IDO, COX2, HO-1, LIF and PD-L1.

In still another aspect, the present invention is directed to a pharmaceutical composition comprising a pharmaceutically acceptable carrier and the regulatory mesenchymal stem cell line or regulatory myeloid-derived suppressor cell line of the present invention.

In additional aspects, the present invention provides methods for inhibiting proliferation of T cells, reducing Th17 or Tc17 differentiation of activated T cells or reducing an inflammatory response, in an individual in need thereof, comprising administering to the individual the regulatory mesenchymal stem cell line, the regulatory myeloid-derived suppressor cell line or the pharmaceutical composition defined herein.

In a further aspect, the present invention is directed to methods for treating or preventing graft-versus-host disease (GVHD), transplanted organ rejection, an autoimmune disease, an inflammatory disease, allergy or an immune-mediated neurodegenerative disorder, comprising administering to an individual in need the regulatory mesenchymal stem cell line, regulatory myeloid-derived suppressor cell line or pharmaceutical composition defined herein.

In yet an additional aspect, the present invention provides a method for improving platelet recovery following organ transplantation in an individual, comprising administering to the individual the regulatory mesenchymal stem cell line, regulatory myeloid-derived suppressor cell line or pharmaceutical composition defined herein.

In yet a further aspect, the present invention provides a kit comprising (a) one or two vessels comprising a composition comprising TGFβ; (b) a vessel comprising a composition comprising an inflammatory agent; (c) a vessel comprising a composition comprising a tryptophan IDO metabolite; and optionally (d) a vessel comprising a primary mesenchymal stem cell line; or (e) a vessel comprising the composition comprising a combination of TGFβ, an inflammatory agent and a tryptophan IDO metabolite as described above; (f) a vessel comprising a composition comprising TGFβ; and optionally (g) a vessel comprising a primary mesenchymal stem cell line; and (h) a leaflet with instructions for application of said composition of (a)-(c) on said cell line of (d) or instructions for application of said composition of (e) and (f) on said cell line of (g).

### BRIEF DESCRIPTION OF DRAWINGS

FIGS. 1A-B show flow cytometry histograms for human and mouse mesenchymal stem or stromal cells (MSCs). Human (A) and Mouse (B) MSCs were characterized by flow cytometry analysis using antibodies against selected markers. hMSCs stained negative for MHC II and the hematopoetic marker CD45 and positive for CD73, CD44, CD105, and CD90. MHC I was also expressed on the cell surface. mMSCs stained negative for MHC II and the hematopoetic markers CD45 and CD11b and positive for CD44, CD105, CD29 and Sca1. Low levels of MHC I were expressed on the cell surface. Histograms represent consistent findings for all the primary cells lines that were used, filled histogram- unstained cells, open histogram-stained cells.

FIGS. 2A-B show that mouse and human MSCs treated with triple combination treatment (TCT) have improved inhibitory effect on T cell proliferation. Mouse/Human MSCs were seeded at 1.2×10⁴ cells/cm² and were treated with TGFβ, IFNγ and kynurenine in different combinations, washed and co-cultured for 4 d with mouse splenocytes/carboxyfluorescin diacetate succinimidyl ester (CFSE) labeled human peripheral blood mononuclear cell (hPBMCs) activated with anti-CD3 antibody, in the ratio of 1/20 respectively. (A) The proliferation of activated mouse splenocytes, analyzed by thymidine incorporation assay. Summary of 4 independent experiments. (B) Proliferation of activated hPBMCs, analyzed using flow cytometry analysis. Summary of independent experiments with hMSCs from 6 different donors. Results are expressed as mean+SE. P value *<0.05, **<0.005. non act T, non-activated T cell; act T, activated T cell; ntMSCs, non-treated MSCs.

FIGS. 3A-F show that TCT-treated mMSCs have a regulatory phenotype. Mouse MSCs were seeded and treated as described in FIG. 2. The expression levels of (A) IDO and (B) iNOS were assessed by qPCR analysis. (C) The expression of MHC I was assessed by flow cytometry. (D) Expression levels of COX2 were assessed by qPCR analysis. (E) PGE2 Quantification was performed using ELISA assays on supernatants. (F) Expression levels of HO-1 were assessed by qPCR analysis. Results are expressed as mean+SE. data is summarized from a minimum of 3 independent experiments. P value *<0.05, **<0.005; n.s., not significant; Kyn, kynurenine; RQ, average relative quantification.

FIGS. 4A-B show that TCT MSCs express higher levels of the regulatory protein PD-L1. Treated/non-treated mMSCs (A) and hMSCs (B) were analyzed using flow cytometry for the expression of Programmed Death Ligand 1 (PD-L1). Results are expressed as mean+SE. data is summarized from at least 3 independent experiments. P value *<0.05, **<0.005.

FIG. 5 shows that kynurenine alone has no effect on the expression of IDO, COX2 IL6 and iNOS. qPCR analysis for the expression levels of IDO, COX2 and IL6, in Kynurenine (Kyn) treated/non-treated (nt) murine MSCs.

FIGS. 6A-C show that the TCT-treated MSCs have more immunoregulatory and less stromal functional phenotype. qPCR for the expression of chemokines (A) hematopoietic niche supporting genes (B) and growth factors (C) in TCT MSCs comparing to non-treated MSCs. Results are expressed as mean +/−SE. data is summarized from 5-8 independent experiments. P value *<0.05, **<0.005, ***<0.0005. RQ, average relative quantification.

FIGS. 7A-E show that TCT-treated hMSCs have a regulatory phenotype. Human MSCs were seeded and treated as in FIG. 1. The expression levels of (A) IDO and (B) iNOS were assessed by qPCR analysis. The expression of (C) MHC I and (D) MHC II were assessed by flow cytometry. (E) Expression levels of COX2 were assessed by qPCR analysis. Results are expressed as mean+SE. data is summarized from a minimum of 3 independent experiments. P value *<0.05, **<0.005; n.s., not significant; Kyn, kynurenine; RQ, average relative quantification.

FIGS. 8A-D show that the effect of kynurenine on MSCs includes AhR activation, decreased IL6 expression/secretion, and enhanced expression of LIF. (A) qPCR analysis for the expression levels of CYP1a1, an AhR dependent gene, in mouse/human MSCs. (B) qPCR analysis for the expression levels of COX2 in mouse/human kynurenine treated MSCs, in the presence or absence of 10 μM CH-223191. (C) Quantification of IL6 secretion was performed using ELISA assay on mMSCs culture medium. qPCR analysis for the expression levels of IL6 in mouse MSCs, in the presence or absence of 10 μM CH-223191. (D) qPCR analysis for the expression levels of LIF in mouse MSCs, in the presence or absence of 10 μM CH-223191. Results are expressed as mean+SE. Data is summarized from a minimum of 3 independent experiments. P value *<0.05, **<0.005. Kyn, kynurenine. RQ, average relative quantification. (New Fig. S5)

FIGS. 9A-E show that kynurenine activates AhR that in turn influence EGFR internalization in mMSCs. (A) qPCR analysis for the expression levels of Cyp1b1, an AhR dependent gene, in triple combination treatment (TCT) mMSCs. (B) qPCR analysis for the expression levels of Cyp1b1 in Kynurenine treated mMSCs, in the presence or absence of 10 μM CH-223191. (C,D) qPCR analysis for the expression levels of Cyp1a1, AhR dependent gene, in Kynurenine treated mMSCs (C) and hMSCs (D) in the presence or absence of 10 μM CH-223191. (E) Flow cytometry analysis of EGFR surface expression on MSCs that were cultured in the presence or absence of 10 μM CH-223191 and treated with Kynurenine. Results are expressed as mean+SE. data is summarized from at least 3 independent experiments. P value *<0.05, **<0.005. RQ, average relative quantification.

FIGS. 10A-D show that TCT-treated mouse or human MSCs inhibit Th17 response. Mouse/Human MSCs were seeded and treated as described in FIG. 2. Quantification of (A) mIL6, (B) mIL17, (C) hIL17, (D) mTNFα and (E) mIFNγ in co-culture supernatants was performed using ELISA assays. Results are expressed as mean+SE. data is summarized from a minimum of 3 independent experiments. P value *<0.05, **<0.005; n.s., not significant; Kyn, kynurenine. non act T, non-activated T cell; act T, activated T cell; ntMSCs, non-treated MSCs.

FIGS. 11A-C show that a single administration of TCT MSCs inhibits acute GVHD and improves survival in the semi allogeneic mouse model. (A) Average GVHD score (Days 13-26, left) and (B) median GVHD score (Day 22, right). Differences between TCT MSCs and untreated mMSCs/control groups are significant. Days 13-26, P value <0.005. Results are expressed as mean +/−SE. (C) Survival curve. Differences between control and TCT mMSCs groups and between TCT and untreated mMSCs groups are significant. Days 0-26, P value <0.05. Data is summarized from 3 independent experiments. non act T, non-activated T cell; act T, activated T cell; ntMSCs, non-treated MSCs.

FIG. 12 depicts a proposed mechanism for the effect of TCT on MSCs. IFNγ and TGFβ in the TCT synergistically up-regulates the expression of iNOS, IDO and PD-L1. Kynurenine in the TCT activates AhR, causing its translocation to the nucleus and suppression of IL6 transcription. Additionally, the AhR-pp60src-EGFR pathway may be activated to induce ERK1/2 phosphorylation, leading to COX2 transcription. COX2 is up-regulated by TGFβ and kynurenine has an additive effect. Other mechanisms specifically induced by TCT include up-regulation of HO-1 and LIF. Altogether, the combination between AhR activation by Kynurenine and the up-regulation of iNOS, IDO, COX2, HO-1, LIF and PD-L1 by IFNγ, TGFβ and Kynurenine enables an ultimate regulatory phenotype for MSCs. As such, TCT MSCs have better inhibitory effect on T cell proliferation as well as Th17 differentiation.

FIGS. 13A-E show that administration of TCT MSCs to a mouse model of acute GVHD results in improved platelet recovery (A) and in increase of the regulatory cytokine IL10 (B) and LIF (C) on day 13 after transplantation, followed by a decrease in IL17 (D) and no change in IFN-γ (E) on day 20 after transplantation.

FIGS. 14A-B show that TCT intravenous administration in a mouse model of acute GVHD affects GVHD score while intramuscular administration affects only skin GVHD. (A) GVHD score; (B) skin GVHD.

FIG. 15 shows a survival graph for mice with acute GVHD treated with two administrations of TCT after transplantation.

### DETAILED DESCRIPTION OF THE INVENTION

As mentioned above, mice or human mesenchymal stem or stromal cells, herein after referred to as mesenchymal stem cells (MSCs), exert powerful anti-inflammatory and immune-suppressive function (Le Blanc, Tammik et al. 2003, Aggarwal and Pittenger 2005, Glennie, Soeiro et al. 2005, Rasmusson, Ringden et al. 2005, Rasmusson, Uhlin et al. 2007, Li, Guo et al. 2008, Sundin, Barrett et al. 2009, Le Blanc and Mougiakakos 2012). MSCs were demonstrated to have a regulatory effect on various immune cells including T cells, B cells, dendritic cells and macrophages. MSCs inhibit T cell proliferation (Aggarwal and Pittenger 2005, Glennie, Soeiro et al. 2005, English, Ryan et al. 2009), and influence T cell differentiation, including inhibition of Th17 and Tc17 cells development (Ghannam, Pene et al. 2010, Luz-Crawford, Noel et al. 2012, Glenn, Smith et al. 2014). The immune-suppressive effects of MSCs are induced by the activation of several key enzymes, such as the nitric oxide producing enzyme—inducible nitric oxide synthase (iNOS), prostaglandin E2 (PGE2) producing enzyme—cyclooxygenase-2 (COX2), carbon monoxide (CO) and biliverdin/bilirubin producing enzyme—heme oxygenase 1 (HO-1) and indoleamine 2,3-dioxygenase (IDO)—which catalyzes the catabolism of tryptophan to kynurenine metabolites. Kynurenine has been found to participate in the immune-regulatory function of IDO (Mellor and Munn 2004, Jasperson, Bucher et al. 2009). Activation of the aryl hydrocarbon receptor (AhR) by kynurenine, negatively regulates dendritic cells (DCs) (Nguyen, Kimura et al. 2010) and generates regulatory T cells (Mezrich, Fechner et al. 2010).

Cell surface expression of inhibitory molecules such as programmed death ligand 1 (PD-L1), also contributes to the immune-suppressive function of MSCs. The importance of each mechanism in the MSCs immune-regulatory response varies between species. IDO up-regulation is described in human MSCs, while iNOS is a key enzyme in mouse MSCs.

Activation of MSCs by IFNγ has the dual effect of enhancing the immunomodulatory response by up-regulation of IDO (Meisel, Brockers et al. 2011) and PD-L1 (Sheng, Wang et al. 2008), and simultaneously activating antigen-presenting cell (APC) functions in MSCs through the elevated expression of MHC II and the acquired ability to process and present antigens (Le Blanc, Tammik et al. 2003, Stagg, Pommey et al. 2006). The addition of TGFβ to IFNγ activated MSCs can reduce their MHC II expression (Romieu-Mourez, Francois et al. 2007).

The present invention is based on the surprising findings that kynurenine activates AhR in MSCs and that together with TGFβ and IFNγ it provides an unexpected synergistic effect on the immune-regulatory phenotype of MSCs.

It has thus been found in accordance with the present invention that mouse or human MSCs (mMSCs and hMSCs, respectively) treated ex vivo with the immune-regulatory triple combination treatment (TCT) of IFNγ, TGFβ and kynurenine inhibits T cell proliferation better than other combinations or untreated MSCs (Example 1). mMSCs and hMSCs treated with the TCT, also referred to herein as TCT mMSCs or TCT hMSCs, respectively, were shown herein to have a regulatory phenotype that involves the expression of PD-L1, which is up-regulated in MSCs in the presence of IFNγ (Sheng, Wang et al. 2008) and the key regulatory enzymes inducible nitric oxide synthase (iNOS), indoleamine 2,3-dioxygenase (IDO) cyclooxygenase-2 (COX2) and heme oxygenase 1 (HO-1). The expression intensity of iNOS was higher than IDO in the murine experiments and lower than IDO in the human experiments, in correspondence with the literature (Ren, Su et al. 2009). (Examples 2 and 3).

In phenotype analysis using quantitative (real time) qRT PCR it was found that TCT mMSCs and hMSCs acquire differentiation commitment to a more immunregulatory and less stromal phenotype. They express higher levels of chemokines attracting immune cells, such as CCL5 (Soria and Ben-Baruch 2008), CXCL1 (Kobayashi 2008), CXCL10 (Dufour, Dziejman et al. 2002) and CXCL11 (Cole, Strick et al. 1998); and lower levels of hematopoietic stem cell niche regulating factors, such as FoxC1 (Omatsu, Seike et al. 2014), CD166 (Chitteti, Bethel et al. 2013, Chitteti, Kobayashi et al. 2013) and stromal cell derived factor 1 (SDF1/CXCL12) (Greenbaum, Hsu et al. 2013). They also express more of the immunosuppressive growth factor VEGF (Gavalas, Tsiatas et al. 2012) and less of the growth factors that are related to cell growth and maintenance in comparison to untreated mMSCs. (Examples 2 and 3).

Example 2 further discloses that PD-L1, iNOS and IDO were synergistically up-regulated by IFNγ and TGFβ, while kynurenine had no effect on the expression of these genes. However, kynurenine upregulated leukemia inhibitory factor (LIF) expression (Example 4), had an additive effect on the upregulation of COX2 and a profound effect on the upregulation of HO-1 (Example 2). These results led us to question the mechanism involved in kynurenine's effect on MSCs. Several papers have reported on the role of nitric oxide in prostaglandin expression (Weinberg 2000, Aisemberg, Vercelli et al. 2007, Kim 2011). Kynurenine's effect on COX2 expression however, was nitric oxide independent and we therefore looked for an alternative mechanism.

AhR is a ligand activated transcription factor, traditionally known as a regulator of drug metabolizing enzymes, but also has an important role in the immune response (Hanieh 2014). Activation of AhR by kynurenine negatively regulates DCs (Nguyen, Kimura et al. 2010) and generates regulatory T cells from naïve T cells (Mezrich, Fechner et al. 2010). AhR plays an essential role in the regulation of the LPS signaling pathway in macrophages (Kimura, Naka et al. 2009). Fritsche et al (Fritsche, Schafer et al. 2007) demonstrated a bifurcated signaling pathway of AhR in response to light, that is, the induction of CYP1A1 through AhR/DRE and the activation of the AhR-pp60src-EGFR pathway to activate ERK1/2 and induce COX2. The interaction between kynurenine and AhR in MSCs has not been studied. It was surprisingly found in accordance with the present invention that kynurenine activates aryl hydrocarbon receptor (AhR) in both mouse and human MSCs and that MSCs express AhR associated genes in response to kynurenine. This expression was abolished in the presence of the AhR antagonist CH-223191. In addition, kynurenine treatment enhanced EGFR internalization in MSCs. This effect, as well as the enhanced expression of COX2 and LIF, was reversed in the presence of the AhR antagonist. These results indicate that the additive effect kynurenine has on the expression of COX2 and LIF in the TCT is related to kynurenine-induced AhR activation (Example 4).

Several reports (Vogel, Goth et al. 2008, Nguyen, Kimura et al. 2010) claim that the expression of IDO can be mediated by AhR signaling in DCs and T cells. Our RT-PCR results clearly show that kynurenine has no effect on the upregulation of IDO or iNOS in the triple combination; we therefore conclude that kynurenine activation of AhR does not induce these enzymes in MSCs.

The Th17/Tc17 response has a central role in autoimmune diseases and graft-versus-host disease (GVHD) pathophysiology. In GVHD, donor naïve T cells differentiate in the presence of TGFβ and IL-6 to Th17 cells and exert a cellular response against host alloantigens. MSCs secrete high levels of IL-6 in the presence of lymphocytes and this may enhance the Th17 response and worsen GVHD (Svobodova, Krulova et al. 2011). It has previously been demonstrated that COX2 and PD-1 pathways are involved in MSC-induced repression of Th17 (Duffy, Pindjakova et al. 2012, Luz-Crawford, Noel et al. 2012). It is shown herein that TCT significantly reduced IL-17 and IL-6 secretion in co-cultures compared to non-treated and immune-regulatory double combination treatment (DCT) mMSCs. It is further shown herein that in the TCT, kynurenine restricts the induced expression and secretion of IL-6 from TGFβ treated mMSCs, and that this effect is AhR dependent (Example 5). These results are supported by Kimura et al., who demonstrated that AhR, along with Stat1 and NF-κB inhibit the promoter activity of IL-6 in macrophages (Kimura, Naka et al. 2009). Moreover, kynurenine-dependent up-regulation of LIF expression in the TCT mMSCs may contribute to the inhibition of the Th17 milieu by its reversal nature to IL-6 (Gao, Thompson et al. 2009, Park, Gao et al. 2011). Based on these results, one may conclude that reduction of IL-6 secretion, together with PD-L1, LIF and COX2 up-regulation; contribute to the inhibition of the Th17 milieu by TCT MSCs.

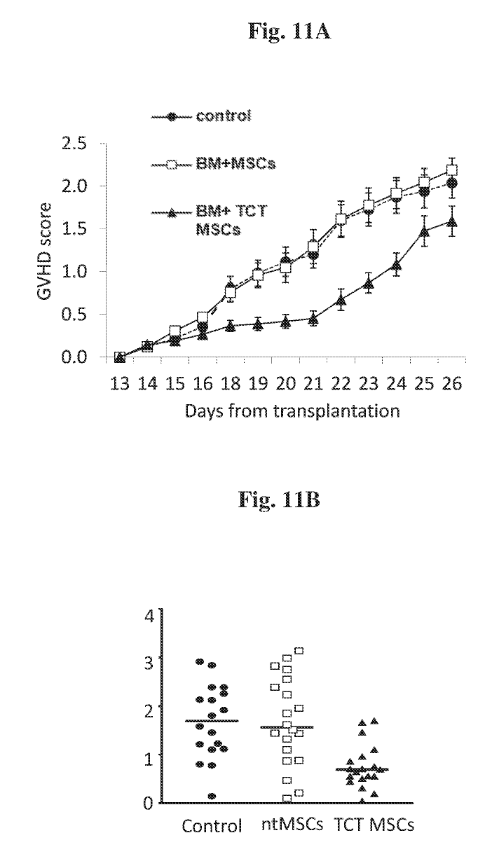

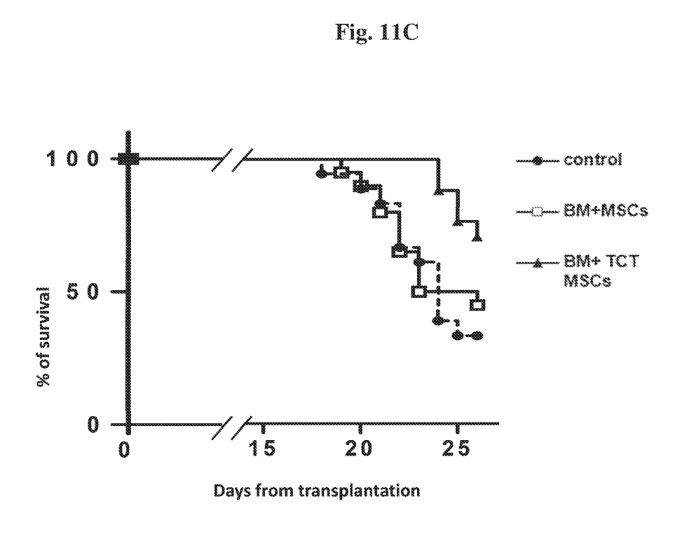

FIG. 11 summarizes a model for the mechanism of regulatory phenotype induction by TCT MSCs: IFNγ and TGFβ, synergistically up-regulate the expression of PD-L1, iNOS and IDO. Kynurenine in the TCT contributes to the regulatory phenotype of mMSCs through the activation of AhR, leading to the suppression of IL-6 secretion and the induction of LIF and the EGFR-COX2 pathway. We also observed an additive effect of kynurenine on the expression of HO-1. AhR activation together with iNOS, IDO, COX2, HO-1, LIF and PD-L1 up-regulation, enables a superior regulatory phenotype for MSCs, allowing these cells to inhibit the proliferation of activated T cells and the differentiation of naïve T cells to Th17.

TCT mMSCs were further examined herein as an immune-regulatory cell therapy in a semi-allogeneic transplanted GVHD mouse model. The involvement of Th17 immune response in GVHD pathogenesis in this model was previously described (Azar, Shainer et al. 2013). It was thus found in accordance with the present invention that TCT mMSCs significantly reduce the GVHD score and improve survival. Importantly, a single administration could attenuate disease symptoms for more than three weeks (Example 6). TCT mMSCs elevated plasma levels of the regulatory cytokines LIF and IL10 and reduced the levels of the Th17 produced cytokine, IL17. Additional administrations provide for longer effects (Example 9). These results indicate that the immune-regulatory properties of TCT MHCs are maintained in vivo. In summary, the findings of the present disclosure indicate that ex-vivo TCT MSCs exhibit a strong regulatory phenotype and as such reduce pathologic inflammation and improve survival in the mouse model.

Another cell type, CD11b(+)Gr1(+) myeloid-derived suppressor cells, hereinafter referred to as myeloid-derived suppressor cells (MDSCs), are an important regulatory innate cell population and have significant inhibitory effect on T cell-mediated responses. In addition to their negative role in cancer development, MDSCs also exert strong regulatory effects on transplantation and autoimmunity. In many transplantation models, such as bone marrow transplant, renal transplant, heart transplant and skin transplant settings, MDSCs accumulate and have inhibitory effect on graft rejection. However, the inducing factors, detailed phenotype and functional molecular mediators of MDSCs are significantly different in various transplant models (Wu, Zhao et al. 2014). MDSCs express all the relevant signal transduction pathways mediating the TCT effect in MSCs as explained herein. Therefore, it is expected that treatment of MDSCs with the TCT of the present invention will induce a stable regulatory phenotype in the MDSCs.

In view of the above, the present invention provides, in one aspect, a composition comprising TGFβ, an inflammatory agent and a tryptophan indoleamine-2,3 dioxygenase (IDO) metabolite, or a pharmaceutically acceptable salt thereof. The terms “inflammatory agent” and “pro-inflammatory agent” are used interchangeably herein.

In a further aspect, the present invention provides a kit comprising (a) one or two vessels comprising a composition comprising TGFβ; (b) a vessel comprising a composition comprising an inflammatory agent; (c) a vessel comprising a composition comprising a tryptophan IDO metabolite; and optionally (d) a vessel comprising a primary mesenchymal stem cell line; or (e) a vessel comprising the composition comprising a combination of TGFβ, an inflammatory agent and a tryptophan IDO metabolite as described above; (f) a vessel comprising a composition comprising TGFβ; and optionally (g) a vessel comprising a primary mesenchymal stem cell line; and (h) a leaflet with instructions for application of said composition of (a)-(c) on said cell line of (d) or instructions for application of said composition of (e) and (f) on said cell line of (g).

In certain embodiments, the inflammatory agent is selected from, but not limited to, IFNγ, TNFα, IL-1 or LPS and the tryptophan IDO metabolite is independently selected from kynurenine, N-formylkynurenine, 3-hydroxykynurenine, 3-hydroxyanthranilic acid or quinolinate. In particular, the inflammatory agent may be IFNγ and independently, the tryptophan IDO metabolite may be kynurenine. In certain embodiments the composition of the invention comprises TGFβ, IFNγ and kynurenine. Where applicable and relevant, a pharmaceutically acceptable salt of any of the above mentioned agents and metabolites is also contemplated.

In certain embodiments, the TGFβ and IFNγ are human or mouse TGFβ and IFNγ, in particular human TGFβ and IFNγ.

The concentration of the TGFβ in the composition may be in the range of 10 to 500 pM; the concentration of the IFNγ in the composition may be in the range of 10 to 1000 ng/ml; and/or the concentration of the kynurenine in the composition may be in the range of 10 to 1000 μM.

The purpose of the composition and its characteristic feature is that, upon its contact with a primary mesenchymal stem cell line or a primary myeloid-derived suppressor cell line it imposes on the cell line an immunosuppressive phenotype, such as the capability of inhibiting T cell proliferation and inhibiting the development of Th17 and Tc17 cells. The term “regulatory” cell is thus used herein to differentiate the naïve, untreated, cell from the TCT treated cell having a stable immunosuppressive phenotype. Methods for assessing T cell proliferation or the levels Th17 and Tc17 cells are readily available to the person skilled in the art. For example, T cell proliferation can be measured as described herein below and the levels Th17 and Tc17 cells can be assessed by labeling PBMCs with antibodies against surface markers for these cells and analyzed in a FACS machine or by assessment of specific cytokines such as IL17 and IL22 by FACS or ELISA assays.

In another aspect, the present invention is directed to a method for obtaining a regulatory mesenchymal stem cell line or a regulatory myeloid-derived suppressor cell line comprising contacting ex-vivo a primary mesenchymal stem cell line or a primary myeloid-derived suppressor cell line with (i) a composition comprising TGFβ, a composition comprising an inflammatory agent and a composition comprising a tryptophan IDO metabolite; or (ii) the composition comprising a combination of TGFβ, an inflammatory agent and a tryptophan IDO metabolite as described above, thereby obtaining the regulatory mesenchymal stem cell line or regulatory myeloid-derived suppressor cell line. In certain embodiments, the compositions of (i) are brought concomitantly in contact with the cell line. The skilled artisan can readily establish that regulatory mesenchymal stem cells have been obtained by measuring the level of certain markers associated with this phenotype, such as but not limited to elevated levels of iNOS, IDO, COX2, HO-1, LIF and/or PD-L1; or by co-culturing the mesenchymal stem cells with spleen cells and monitoring T cell proliferation upon activation or Th17/Tc17 differentiation for example by measuring cytokine secretion.

In certain embodiments, the inflammatory agent in (i) or (ii) is selected from IFNγ, TNFα, IL-1 or LPS and the tryptophan IDO metabolite in (i) or (ii) is selected from kynurenine, N-formylkynurenine, 3-hydroxykynurenine, 3-hydroxyanthranilic acid or quinolinate. In particular, the inflammatory agent in (i) or (ii) may be IFNγ and independently, the tryptophan IDO metabolite may be kynurenine.

In certain embodiments, the primary mesenchymal stem cell line or primary myeloid-derived suppressor cell line is contacted with (i) a composition comprising TGFβ, a composition comprising IFNγ and a composition comprising kynurenine; or (ii) a composition comprising TGFβ, IFNγ and kynurenine. In certain embodiments, the compositions of (i) are brought concomitantly in contact with the cell line. The concentration in (i) or (ii) of the TGFβ may be in the range of 10 to 500 pM; the concentration in (i) or (ii) of the IFNγ may be in the range of 10 to 1000 ng/ml; and/or the concentration in (i) or (ii) of the kynurenine may be in the range of 10 to 1000 μM. In certain embodiments, the TGFβ and IFNγ used in the method are human or mouse TGFβ and IFNγ, in particular human TGFβ and IFNγ. The term “primary mesenchymal stem cell line” is used interchangeably herein with the term “primary mesenchymal stem cells”.

In certain embodiments, the cell line is contacted with TGFβ prior to the contacting with the compositions of (i) or composition of (ii). The cell line may be incubated with the compositions of (i) or composition of (ii) for about 8 to 48 hrs, or the cell line may be incubated with TGFβ for about 8 to 48 hrs prior to incubation with the compositions of (i) or composition of (ii) for about 8 to 48 hrs.

In certain embodiments, the primary mesenchymal stem cell line and primary myeloid-derived suppressor cell line is a human primary mesenchymal stem cell line and a human primary myeloid-derived suppressor cell line, respectively.

In yet another aspect, the present invention provides a regulatory mesenchymal stem cell line or a regulatory myeloid-derived suppressor cell line obtained according to the methods defined herein.

In still another aspect, the present invention provides a regulatory mesenchymal stem cell line or a regulatory myeloid-derived suppressor cell line characterized by elevated levels of a cell marker selected from iNOS, IDO, COX2, HO-1, LIF and PD-L1.

The term “elevated level” as used herein refers to a level of a cell marker in a TCT treated MSC or MDSC that is statistically significantly higher relative to a native, untreated, naïve MSC or MDSC, respectively, derived from the same species or such MSCs or MDSCs treated with DCT. Methods for assessing the levels of iNOS, IDO, COX2, HO-1, LIF and PD-L1 are readily available to the person skilled in the art. For example, expression levels of the relevant genes may be measured using qPCR or proteins expressed on the surface of the cells can be measured using fluorescence labeled specific antibodies and FACS, as described herein in the Examples.

In certain embodiments, the regulatory mesenchymal stem cell line or myeloid-derived suppressor cell line of the present invention is derived from human cell lines.

In a further aspect, the present invention is directed to a pharmaceutical composition comprising a pharmaceutically acceptable carrier and the regulatory mesenchymal stem cell line or regulatory myeloid-derived suppressor cell line of the present invention.

It has been found in accordance with the present invention that the total GVDH score of an individual having GVHD can be reduced by intravenous (IV) injection of the regulatory mesenchymal stem cells of the present invention and that the skin GVHD score can be significantly reduced by intramuscular (IM) injection of the cells (Example 8). Thus, in certain embodiments, the pharmaceutical composition is formulated for intravenous or intramuscular injection.

In yet further aspects, the present invention is directed to the regulatory mesenchymal stem cells or cell line or regulatory myeloid-derived suppressor cells/cell line obtained according to the methods defined herein and/or characterized by elevated levels of one or more cell markers as defined herein above, the pharmaceutical composition comprising the regulatory mesenchymal stem cells/stem cell lines or regulatory myeloid-derived suppressor cells/cell line, for use in inhibiting or reducing T cell proliferation and/or reducing Th17 or Tc17 differentiation of activated T cells in an individual, or for reducing an inflammatory response, such as an exaggerated or uncontrolled inflammatory response.

In an additional aspect, the present invention provides a method for inhibiting or reducing T cell proliferation and/or reducing Th17 or Tc17 differentiation of activated T cells in an individual or for reducing an inflammatory response, such as an exaggerated or uncontrolled inflammatory response, in an individual in need thereof, comprising administering to the individual the regulatory mesenchymal stem cells as defined herein.

In certain embodiments, the regulatory mesenchymal stem cells of the present invention are derived from allogeneic primary mesenchymal stem cell lines.

The regulatory mesenchymal stromal cells/stem cell lines obtained according to the methods defined herein, the pharmaceutical composition comprising the regulatory mesenchymal stromal cells/stem cell lines and the method of the invention may be for use in, but is not limited to, treating or preventing graft-versus-host disease (GVHD), transplanted organ rejection, an autoimmune disease, an inflammatory disease, allergy or an immune-mediated neurodegenerative disorder.

In certain embodiments, the treatment results in a reduced total GVHD, for example when the regulatory mesenchymal stem cells or regulatory myeloid-derived suppressor cells of the present invention are administered via IV injection. In certain embodiments, the treating results in a reduced skin GVHD score, for example when the regulatory mesenchymal stem cells or regulatory myeloid-derived suppressor cells of the present invention are administered via IM injection.

The term “reduced GVHD score” as used herein refers to a lower GVHD score of a patient at a given time point after treatment started as compared with the GVHD score of the same patient before treatment started or as compared with an average GVHD score of untreated GVHD patients.

The treatment may be prophylactic in the sense that the regulatory mesenchymal stem cells or regulatory myeloid-derived suppressor cells are administered before transplantation or before signs of GVHD appear, or it may be therapeutic in the sense that the regulatory mesenchymal stem cells or regulatory myeloid-derived suppressor cells are administered after signs of GVHD appear (e.g. as measured total or skin GVHD score).

It has further been found in accordance with the present invention that repeated administration of the regulatory mesenchymal stem cells described herein to a GVHD model in mice 22 days after transplantation and the first administration of the cells results in significantly improved survival of the mice. Thus, in certain embodiments, the treating comprises administration of said regulatory mesenchymal stem cells, regulatory myeloid-derived suppressor cells or pharmaceutical composition on at least two separate occasions. Both administrations may be done by means of IV injection or one administration may be done by means of IV injection and the other by means of IM injection. In particular, the first administration may be done IV and the second administration may be done IM. The period between the first and the second administration may be determined by the clinical signs of GVHD. Alternatively, the period between the first and the second administration may be in the range of 15-100 days after transplantation. (100 days is the definition of acute GVHD).

It has also been found in accordance with the present invention that treatment of mice having GVHD with the regulatory mesenchymal stem cells as defined herein improves platelet recovery (Example 7). Thus, in certain embodiments, the regulatory mesenchymal stem cell line, regulatory myeloid-derived suppressor cell line or pharmaceutical composition of the present invention may be for use in improving platelet recovery following hematopoietic stem cell transplantation. The term “improving platelet recovery” as used herein refers to elevated platelets count in a patient after treatment relative to platelets counts in the same patient before treatment or as compared with an average platelet count of untreated GVHD patients.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

As used herein, the terms “subject” or “individual” or “animal” or “patient” or “mammal,” refers to any subject, particularly a mammalian subject, for whom diagnosis, prognosis, or therapy is desired, for example, a human.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

According to the present invention, any pharmaceutically acceptable salt of the active agent can be used. Examples of pharmaceutically acceptable salts include, without being limited to, the mesylate salt, the esylate salt, the tosylate salt, the sulfate salt, the sulfonate salt, the phosphate salt, the carboxylate salt, the maleate salt, the fumarate salt, the tartrate salt, the benzoate salt, the acetate salt, the hydrochloride salt, and the hydrobromide salt.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

The determination of the doses of the active ingredient to be used for human use is based on commonly used practices in the art, and will be finally determined by physicians in clinical trials. An expected approximate equivalent dose for administration to a human can be calculated based on the in vivo experimental evidence disclosed herein below, using known formulas (e.g. Reagan-Show et al. (2007) Dose translation from animal to human studies revisited. The FASEB Journal 22:659-661). According to this paradigm, the adult human equivalent dose (mg/kg body weight) equals a dose given to a mouse (mg/kg body weight) multiplied with 0.081.

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques. Alternatively, the term “about” as used herein means that values of 10% or less above or below the indicated values are also included.

The invention will now be illustrated by the following non-limiting examples.

### EXAMPLES

### Material and Methods

Mice. Female 8- to 11-week-old C57BL/6, BALB/c, and (C57BL/6×BALB/c) F1 mice (Harlan Laboratories, Jerusalem, Israel) were used. The study was conducted under appropriate conditions and was approved by the Institutional Animal Care and Use Committee of the Hebrew University of Jerusalem in accordance with national laws and regulations for the protection of animals.

MSCs isolation and culture. Mouse MSCs (mMSCs) were obtained from the bone marrow (BM) of C57BL/6 mice. BM cells were cultured at a concentration of 1*10⁶ cells/ml in Mesencult basal medium supplemented with MSC stimulatory supplements (StemCell Technologies, Vancouver, BC), at 37° C. with 10% CO₂. After 2-3 passages, the cells were harvested and CD11b⁺ cells were depleted by EasySep® cell separation kit (StemCell Technologies, Vancouver, BC). Cells from 3 different lines were used for experiments after 5-20 passages.

Human MSCs (hMSCs). Bone marrow for hMHC was obtained from 17 adult healthy donors, males and females, following informed consent and under institutional Helsinki approval; hMSCs were cultured as previously described and the cells were used for experiments after 3-6 passages (Resnick, Barkats et al. 2013).

The phenotype of mMSCs and hMSCs was assessed by flow cytometry (FACS) analysis.

TCT MSCs were treated with 40pM hTGFβ (PeproTech, Israel) for 24 h, and then with a cocktail of 40 pM hTGFβ, 100 ng/ml h/mIFNγ (PeproTech, Israel) and 150 μM Kynurenine (Sigma, Switzerland) for an additional 24 h before they were used for experiments.

T-cell proliferation assays. mMSCs were plated at a concentration of 5*10⁴ cells/well in 96-well flat bottom plates with RPMI 1640 medium supplemented with 10% FCS, 1% penicillin/streptomycin, and 1% L-glutamine (Biological Industries, Beit Haemek, Israel). A total of 1*10⁶ splenocytes were plated in the presence of or in the absence of previously plated MSCs at a 20:1 ratio and activated with anti CD3 antibodies (Biolegend, USA). After 3 days of co-culture, cells were pulsed for 16 additional hours with ³H-thymidine at 1 μCi/well (PerkinElmer, Boston Mass., USA) and harvested. ³H-thymidine incorporation was measured using Top Count NXT (PerkinElmer, UK).

hMSCs were plated at a concentration of 2*10⁴ cells/well in 24-well flat bottom plates with RPMI 1640 medium supplemented with 10% FCS, 1% penicillin/streptomycin, and 1% L-glutamine A total of 4*10⁵ carboxyfluorescin diacetate succinimidyl ester (CFSE) labeled human peripheral blood mononuclear cell (hPBMCs) were plated in the presence of or in the absence of previously plated hMSCs at a 20:1 ratio. PBMCs were activated with anti CD3 antibody (Biolegend, USA) for 4 days. CFSE levels on the cells were determined using FACS analysis.

FACS analysis. The phenotype of the MSCs was analyzed by FACS using anti-CD29-PB and anti-hCD45-FITC (Biolegend), anti-CD11b-APC, anti-CD44-PE and anti-mCD45-FITC (SouthernBiotech), anti-CD105-PB, anti-CD73-PE, anti-CD90-PE and anti-Sca1-PE (eBioscience), anti-CD44-V450, anti-CD105-APC, anti-HLA DR-APC and anti-HLA ABC-PE (BD Bioscience) antibodies. The expression level of PD-L1 in mMSCs was analyzed by FACS with anti-B7-H1-PE (PD-L1) (eBioscience). To analyze EGFR internalization in kynurenine treated mMSCs, mMSCs were cultured in the presence of 10 μM CH223191 (Merck-Millipore) for 16 hours and then kynurenine was added for an additional 5 hours. EGFR expression was analyzed by FACS using anti-EGFR-PE (Bioss). FACS was performed using the aMACSQuant analyzer (Miltenyi Biotech, Germany) and the data was analyzed using FCS Express V3 software.

ELISA. Quantification of PGE2 in supernatants collected from treated mMSCs and from co-cultures with splenocytes after 72 hours of culture, was performed by competitive enzyme-linked immunosorbent assay (ELISA) technique using a commercially available ELISA kit (R&D Systems, Minn., USA). IL-6, IL-17A and IFNγ in supernatants collected from treated mMSCs and/or from co-cultures with splenocytes/PBMCs were quantified using the ELISA Ready SET Go kits (eBioscience, San Diego, Calif., USA), according to the manufacturer's instructions. All determinations were made in triplicates.

RNA extraction and PCR analysis. Total cellular RNA was extracted using RNeasy® Mini Kit columns (QIAGEN) according to the manufacturer's protocols. 1 μg of total RNA was used to synthesize cDNA using High-Capacity cDNA kit (Applied Biosystems) following the supplier's instructions. Detection of transcript levels of iNOS, IDO, COX2, IL-6, CYP1a1 and CYP1b1, were performed using the TaqMan Gene Expression Assay Kit (Applied Biosystems), using HPRT-1 as a reference. All primers were purchased from Applied Biosystems. Real-Time PCR reactions were conducted using StepOne Plus (Applied Biosystems). Data was analyzed by StepOne Software version 2.2 (Applied Biosystems).

Comprehensive phenotype analysis was performed using quantitative RT-PCR (qRT-PCR) at GGA (Galil Genetics Analysis Ltd., Katzrin, Israel). qRT-PCR was performed in 96.96 dynamic array integrated fluidic circuits (IFCs) (Fluidigm, San Francisco, Calif.) using the EvaGreen DNA-binding dye (Biotium Inc., Hayward, Calif.). Expression values were normalized to the levels of the GAPDH housekeeping gene. Results were analyzed using the Fluidigm data collection software v.3.0.0 and the Fluidigm real-time PCR analysis software v.3.0.2. Fold change was calculated as the average relative quantification (RQ=2^{-ΔΔCT}) values for samples from eight independent experiments.

GVHD mouse model. A semi-allogeneic GVHD mouse model was used as previously described (Azar, Shainer et al. 2013). Briefly, recipient F1 mice received lethal whole-body irradiation and were reconstituted with 8*10⁶ donor C57BL/6 BM cells and 1*10⁷ spleen cells the following day. Concurrently, 1*10⁶ treated/untreated mMSCs were injected intravenously (IV). For GVHD evaluation, mice were monitored daily for weight loss, diarrhea, ruffled skin, and survival (Samuel, Azar et al. 2008). GVHD score, based on all of the aforementioned factors (rated on a scale of 0-4), was calculated (Przepiorka, Weisdorf et al. 1995).

Statistical Analysis. The analysis of GVHD data was performed using MedCalc Software, ANOVA. The analysis of survival data was performed using Kaplan-Meier curves. In vitro data was analyzed using the student t test, with P value <0.05 considered statistically significant.

### EXAMPLE 1

### TCT of Mouse/Human MSCs Improves their Inhibitory Effect on T Cell Proliferation

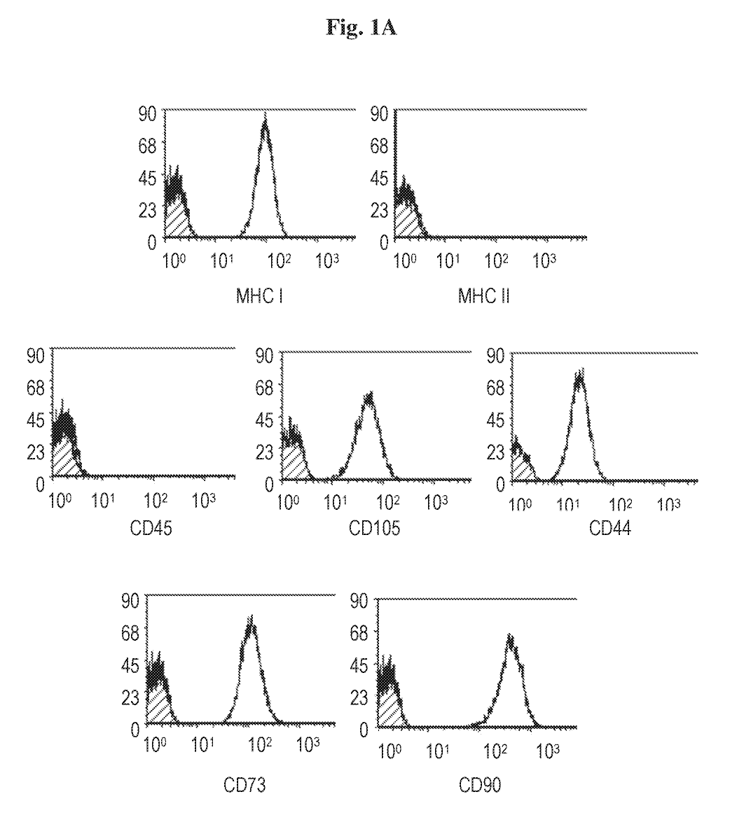

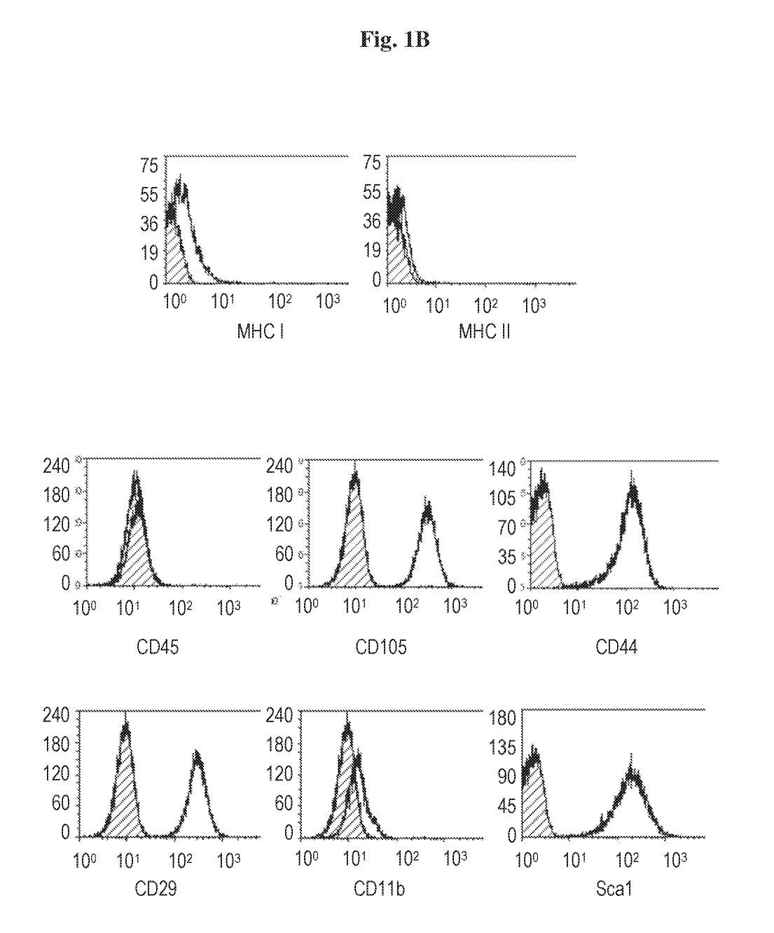

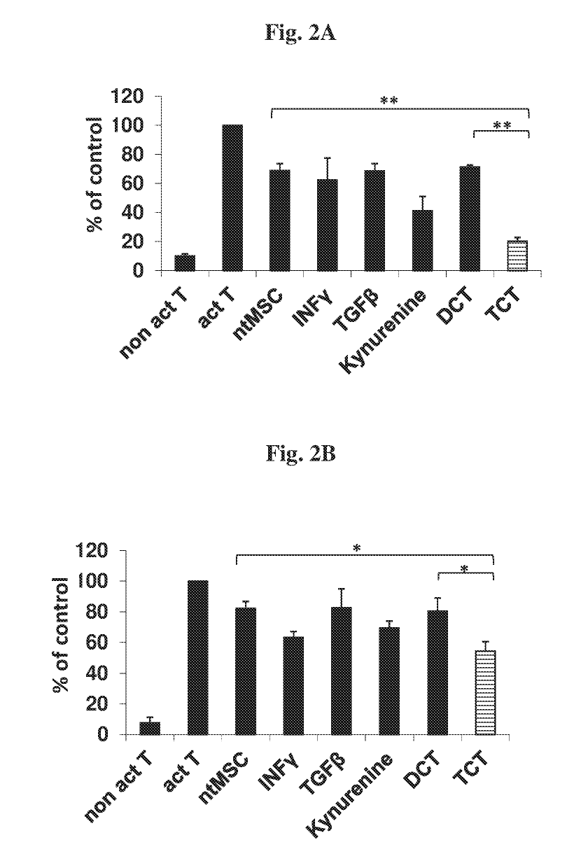

In order to improve MSCs immune-regulatory function, the effects of different molecules on murine/human MSCs were examined and a triple combination treatment (TCT) for the cells was established using IFNγ, TGFβ and kynurenine. Mouse and human MSCs were obtained from C57BL/6 mice and healthy donor BM respectively. MSCs were characterized using FACS analysis (FIGS. 1A; B). The cells were then treated with different combinations of regulatory inducers. To test the immunregulatory function of treated cells, their effect on the proliferation of activated T cells was analyzed. Treated/non-treated MSCs were co-cultured with anti-CD3 activated mSplenocytes/hPBMCs in the ratio of 1:20. Mouse splenocytes proliferation was assessed using thymidine incorporation assay and human PBMCs proliferation was analyzed using CFSE FACS analysis. In both murine and human assays, TCT MSCs inhibited proliferation significantly better than non-treated MSCs or other tested combinations (FIGS. 2A; B respectively).

### EXAMPLE 2

### TCT Mouse MSCs Exhibit a Regulatory Phenotype

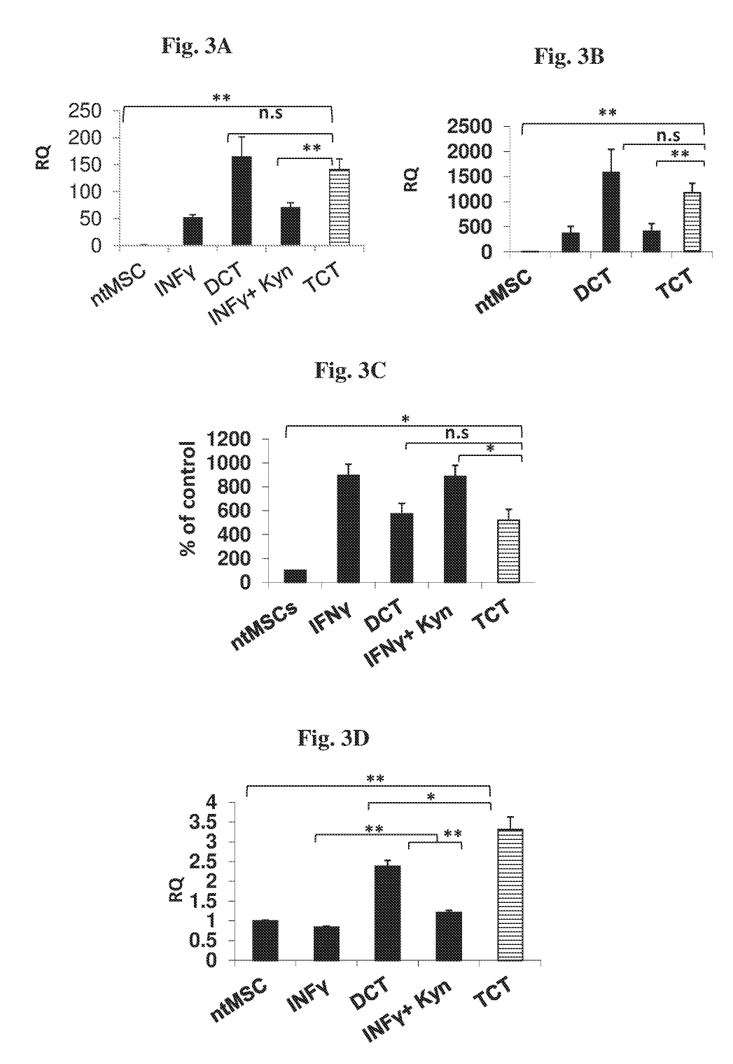

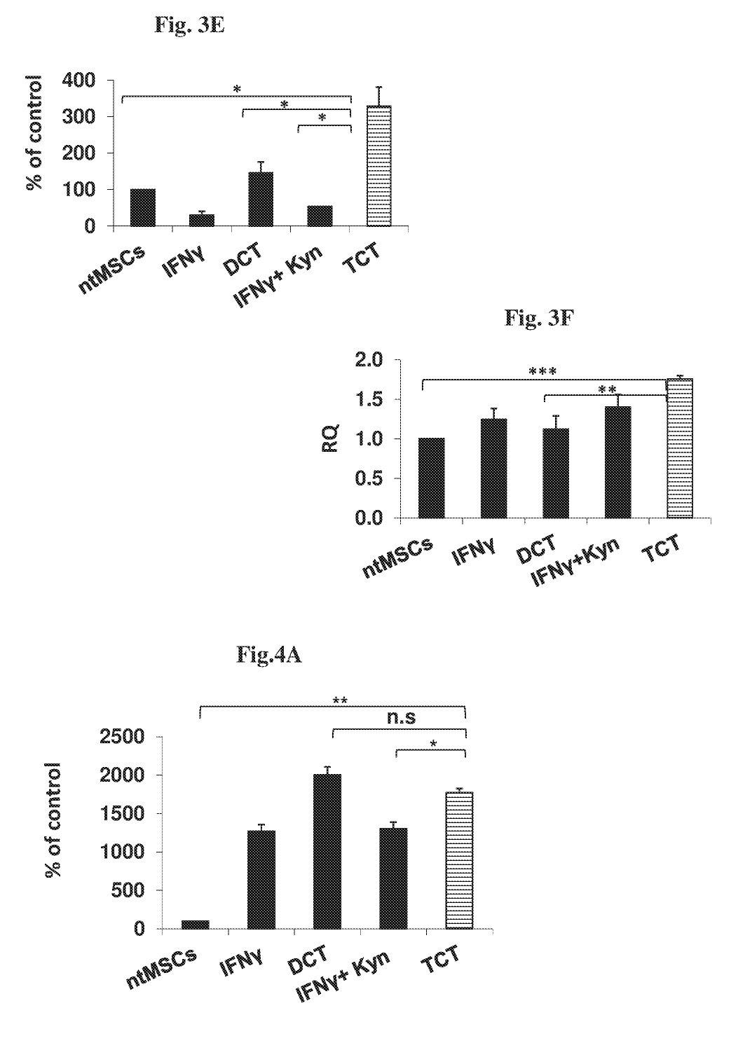

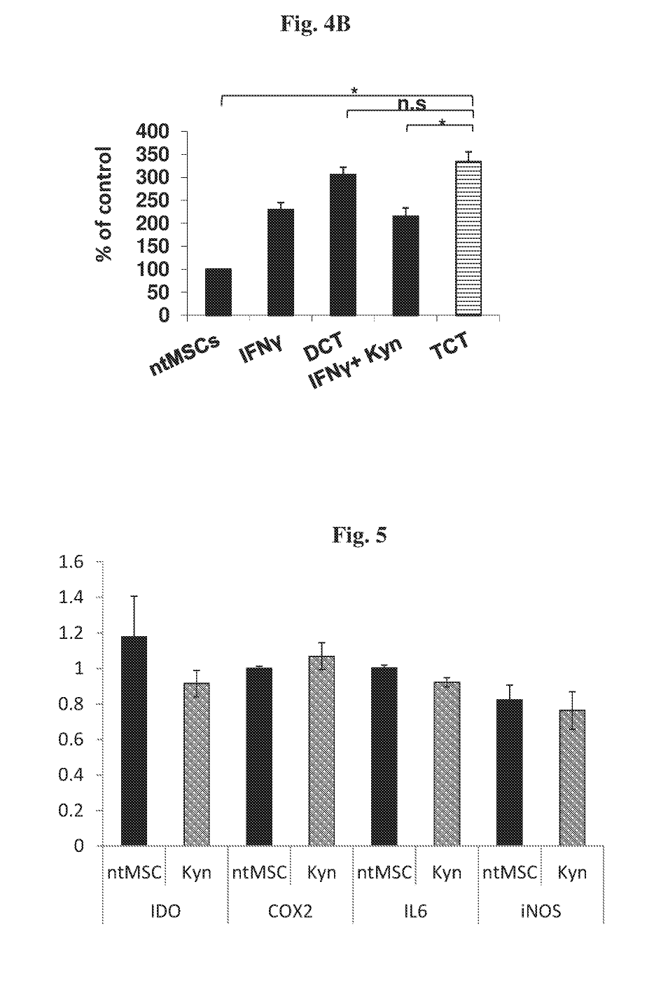

To get a better understanding of the immune-regulatory mechanisms activated in TCT mMSCs, the effect of the treatment on expression levels of different molecules previously described as participating in MSCs regulatory function was examined (Plumas, Chaperot et al. 2005, Ryan, Barry et al. 2007, Sato, Ozaki et al. 2007, Ren, Zhang et al. 2008, Sheng, Wang et al. 2008, Ge, Jiang et al. 2010, Francois, Romieu-Mourez et al. 2011, Li, Ren et al. 2012, Luz-Crawford, Noel et al. 2012, Chinnadurai, Copland et al. 2014). Real time PCR analysis of IDO and iNOS expression levels show synergistic elevation in mRNA levels of both IDO (FIG. 3A) and iNOS (FIG. 3B) for TCT and for the dual combination treatment (DCT) with IFNγ/TGFβ and without kynurenine. Cell surface expression of PD-L1, a regulatory molecule involved in peripheral tolerance, examined by FACS, was also elevated in the DCT and in the TCT (FIG. 4A). Low levels of MHC I expression allow MSCs to remain non-immunogenic and therefore prolong their function in allogeneic settings. IFNγ treatment induces up-regulation of MHC I expression (Francois, Romieu-Mourez et al. 2009) (FIG. 3C). The addition of TGFβ reduced the elevated expression of MHC I both in the TCT and DCT (FIG. 3C). MHC II expression in mMSCs was exceptionally low, and was not elevated in any of the treatments (data not shown).

Examination of the above mechanisms did not reveal any contribution of kynurenine to the improved inhibitory effect of the TCT mMSCs on T cell proliferation (FIG. 5). Therefore, the mRNA expression levels of other immune regulatory enzymes were investigated. While COX2 expression was increased by TGFβ, the addition of kynurenine had significant additive effects on the up-regulation of this enzyme (FIG. 3D). Similar results were obtained for the secretion of the COX2 metabolite, PGE2 (FIG. 3E). IFNγ had no effect on COX2 expression and PGE2 secretion. HO-1, an immune-regulatory enzyme, was also significantly up-regulated by the addition of kynurenine to the DCT (FIG. 3F).

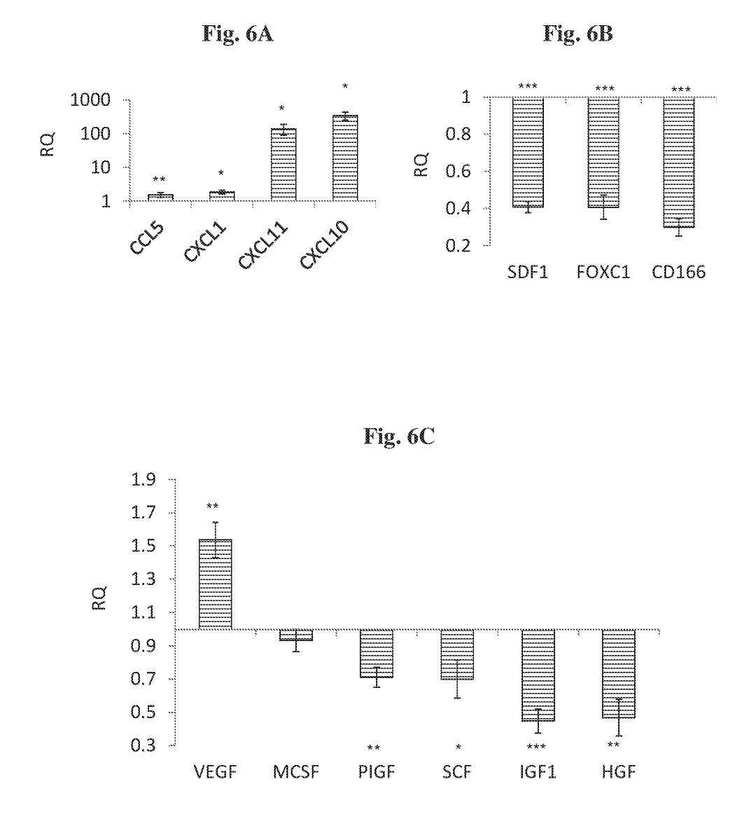

To characterize the phenotype and function of TCT mMSCs, a comprehensive expression analysis using qRT PCR was conducted. The TCT mMSCs significantly expressed higher levels of chemokines, which attract immune cells, and lower levels of factors related to the regulation of the hematopoietic stem cell niche, such as FoxC1, CD166 and Stromal cell Derived Factor 1 (SDF1), in comparison to non-treated mMSCs (previous FIGS. 6A, B respectively). Moreover, TCT mMSCs significantly expressed more of the immunosuppressive growth factor VEGF and less growth factors related to cell growth and maintenance (previous FIG. 6C). These results indicate the differentiation commitment of the TCT mMSCs to an immunosuppressive and less stromal phenotype.

Altogether, these results confirm that TGFβ, IFNγ and kynurenine treatment produce a regulatory phenotype on mMSCs.

### EXAMPLE 3

### TCT-Treated Human MSCs Exhibit a Regulatory Phenotype

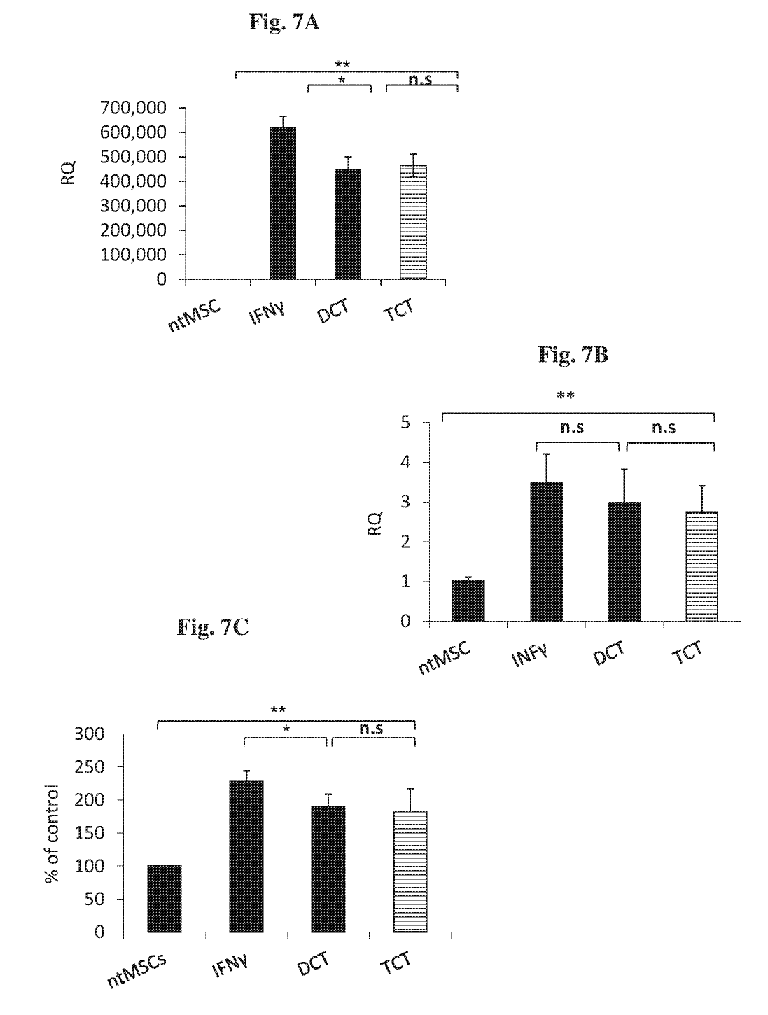

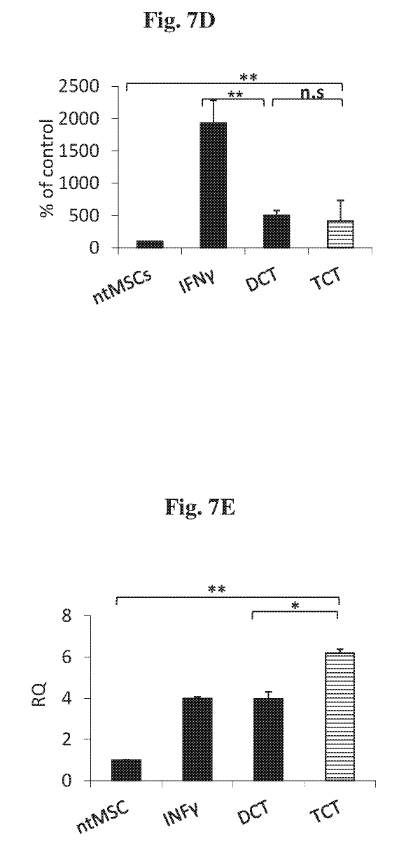

The influence of TCTs on hMSCs was examined to assess the clinical potential of this treatment. qRT-PCR analysis was performed to determine the expression levels of IDO and iNOS in hMSCs. The results on hMSCs correspond with the murine model, with mRNA levels significantly elevated for both IDO (FIG. 7A) and iNOS (FIG. 7B) in the TCT and DCT, when compared to non-treated hMSCs. PD-L1 cell surface expression also had a similar pattern on hMSCs as on mMSCs (FIG. 4B). TGFβ reduced the IFNγ induced expression of both MHC I and MHC II (FIGS. 7C and D, respectively) in the TCT and DCT. The addition of kynurenine significantly elevated COX2 mRNA expression in hMSCs (FIG. 7E). Altogether, these results indicate that, similar to mMSCs, hMSCs gain a regulatory phenotype under the triple combination treatment.

### EXAMPLE 4

### Kynurenine Activates the Aryl Hydrocarbon Receptor both in Mouse and Human MSCs

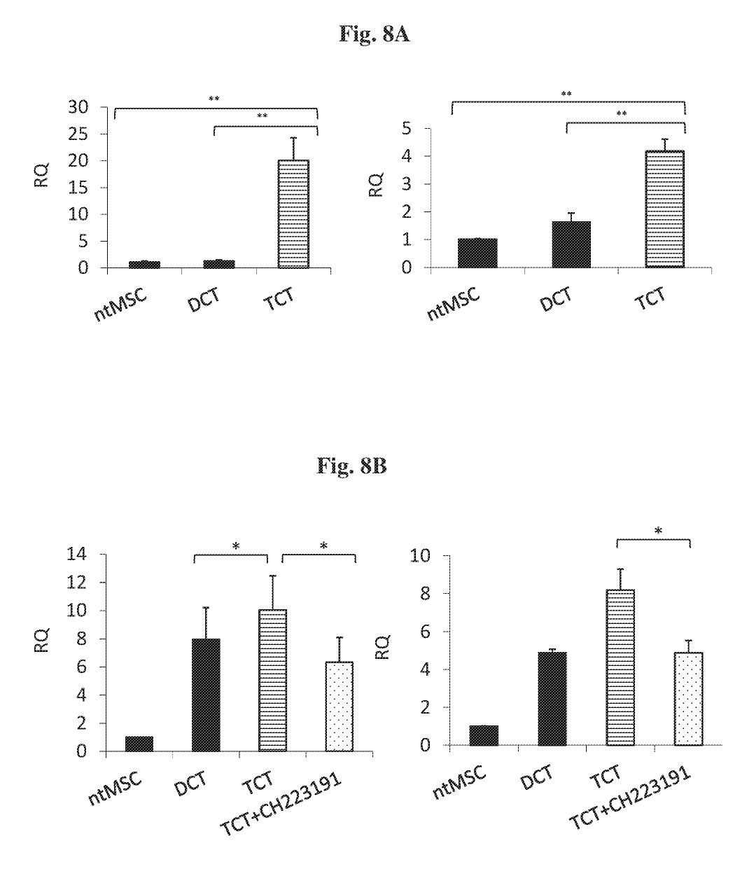

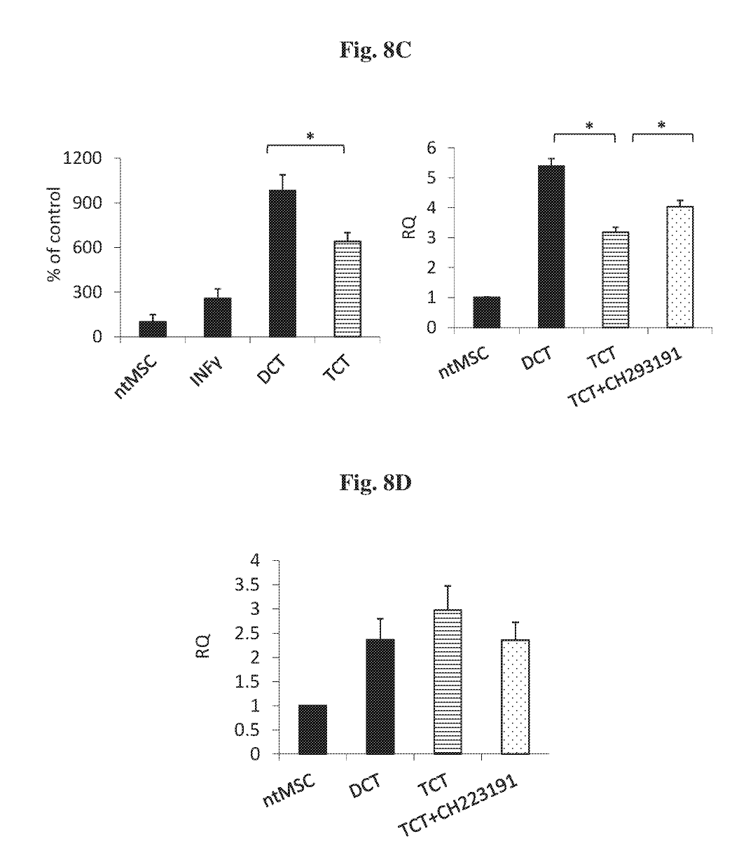

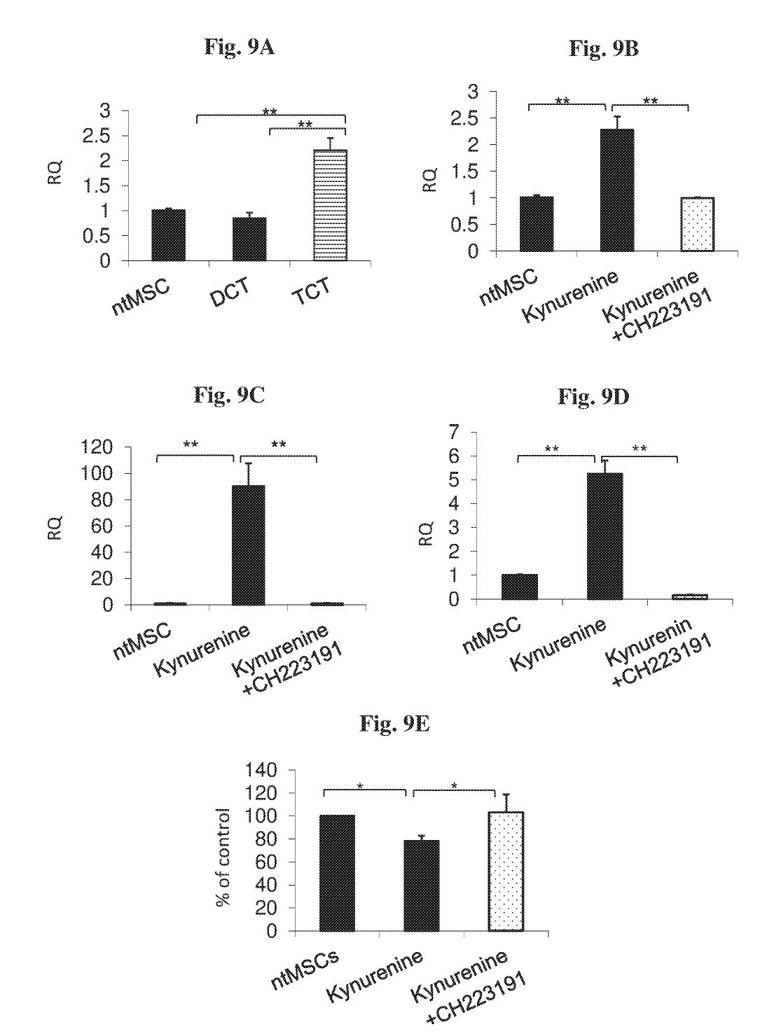

Our combined results strongly suggest that kynurenine plays an essential role in the ability of TCT MSCs to inhibit T cell activation. It was recently demonstrated that kynurenine regulates the immune properties of DCs by activating the transcription factor AhR (Nguyen, Kimura et al. 2010). To evaluate whether kynurenine activates AhR in mMSCs, the mRNA of treated/non-treated MSCs were tested for the expression level of two AhR dependent genes, Cyp1a1 and Cyp1b1. A significant up-regulation of Cyp1a1 in the TCT compared with the DCT in both mouse and human MSCs was found (FIG. 8A). Similar results were obtained for Cyp1b1 expression in mMSCs (previous FIG. 9A), indicating that kynurenine activates AhR in MSCs. To ascertain that the up-regulation of these genes is AhR dependent, MSCs were treated with kynurenine for 24 hours in the presence/absence of the AhR antagonist CH-223191. The up-regulation of Cyp1a1 by kynurenine is inhibited in the presence of the antagonist both in mouse and human MSCs (previous FIGS. 9B, C). Similar results were obtained for Cyp1b1 expression in mMSCs (previous FIG. 9D).

Fritsche et al (Fritsche, Schafer et al. 2007) demonstrated that AhR induces COX2 expression, which is also associated with signal transduction involving EGFR internalization. To investigate whether kynurenine activates the AhR-EGFR-COX2 pathway in MSCs, mMSCs were cultured in the presence of kynurenine with/without CH-223191 pre-treatment. EGFR surface expression is significantly reduced in mMSCs upon kynurenine treatment (previous FIG. 9E). The addition of CH-223191 restored EGFR expression on the cell surface, indicating that EGFR internalization by kynurenine is AhR dependent. In mouse and human MSCs, upregulation of COX2 by kynurenine is AhR dependent (FIG. 8B). Kimura et al (Kimura, Naka et al. 2009) demonstrated, using macrophages, that AhR activation can lead to reduced expression of IL-6. In light of this, the influence of the TCT on MSCs secretion of IL-6 was investigated. The DCT elevated IL-6 secretion (FIG. 8C) as well as mRNA expression in mMSCs, whereas the addition of kynurenine restricted this elevation. The addition of AhR antagonists to the triple combination partially reversed the effects of kynurenine (FIG. 8C), indicating the participation of AhR activation in the inhibition of IL-6 transcription. Moreover, in the TCT cells, enhanced expression of the leukemia inhibitory factor (LIF) (FIG. 8D), which functions as a polar opposite of IL-6 in CD4+ T cells (Gao, Thompson et al. 2009, Park, Gao et al. 2011), is also AhR dependent.

These results strongly suggest that, in the TCT MSCs, the additive effect kynurenine has on the expression of COX2 and LIF and its inhibitory effect on IL-6 secretion are related to kynurenine induced AhR activation.

### EXAMPLE 5

### TCT-Treated MSCs Inhibit Secretion of Inflammatory Cytokines in Co-Culture with Activated T Cells

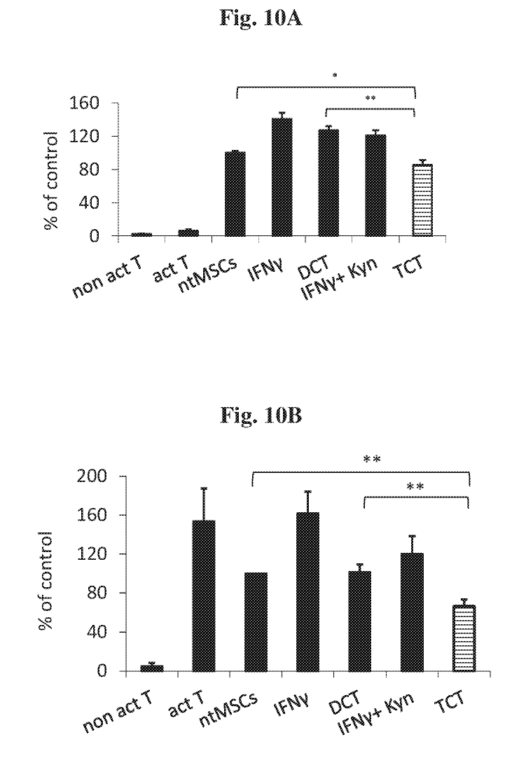

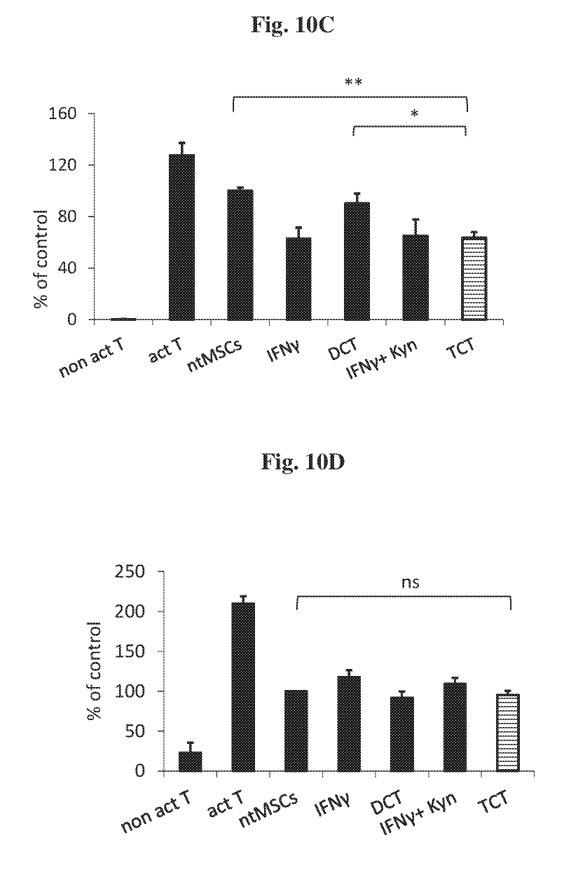

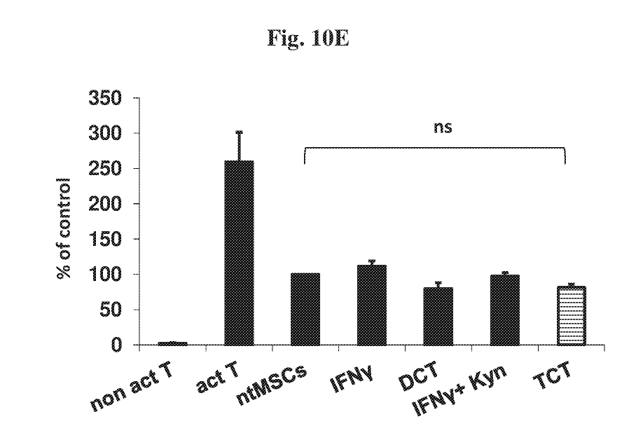

The effects of TCT MSCs on T cell activation and differentiation in co-culture were investigated. Mouse splenocytes or human PBMCs were activated with anti-CD3 antibodies in the presence of treated/non-treated MSCs. The level of secreted cytokines was measured from the culture medium using ELISA assays. TCT mMSCs had significantly reduced IL-6 secretion compared to the non-treated and DCT mMSCs (FIG. 10A). IL-6 has pro-inflammatory properties, and along with TGFβ can induce Th17 differentiation and enhance GVHD in MSCs treated patients (Svobodova, Krulova et al. 2011). When the influence of TCT MSCs on Th17 differentiation was examined, we observed a significant reduction of IL-17 secretion from the activated T cells both in mouse and human co-culture assays (FIGS. 10B, C respectively). The inhibitory effect of TCT MSCs on the secretion of the inflammatory cytokines IFNγ and TNFα was similar to the effect of non-treated MSCs (FIGS. 10D, E respectively). These results indicate that the TCT MSCs have a unique inhibitory effect on Th17 differentiation of T cells, while preserving the inhibitory effect on Th1 response.

### EXAMPLE 6

### The TCT-Treated mMSCs Inhibit Acute GVHD and Improve Survival in Semi-Allogeneic BM Transplantation Mouse Model

Based on the promising in vitro results, we decided to test the clinical potential of the TCT mMSCs was tested in-vivo. Increasing evidence indicates the major role of IL17-producing T cells in GVHD pathogenesis (Dander, Balduzzi et al. 2009, Kappel, Goldberg et al. 2009, Ratajczak, Janin et al. 2010). In addition, acute GVHD therapy using hMSCs has been clinically tested and was recently approved for pediatric allogeneic transplant recipients in Canada and New Zealand (Newell, Deans et al. 2014). We therefore tested the immunosuppressive effect of the TCT mMSCs on GVHD prophylaxis in a murine model. F1 mice underwent whole-body irradiation followed by semi-allogeneic bone marrow transplantation (BMT) from C57BL/6 donor mice. Two groups of mice received a single IV administration of 1*10⁶ treated/non-treated mMSCs on the day of BMT, whereas the control group received BMT alone (Figure Scheme I).

GVHD scores were significantly lower in mice receiving TCT MSCs than in mice receiving non-treated MSCs or in control mice (FIG. 11A left). The distribution of GVHD scores, at day 22 (FIG. 10B right) shows the non-consistent results of non-treated MSCs in-vivo Importantly, TCT MSCs also significantly improved mice survival (FIG. 11B). These results confirm that the TCT MSCs are better modulators of allogeneic activation in-vivo in comparison to non-treated MSCs.

### EXAMPLE 7

### TCT Treatment Improves Platelets Recovery and has a Regulatory Effect on Plasma Cytokines

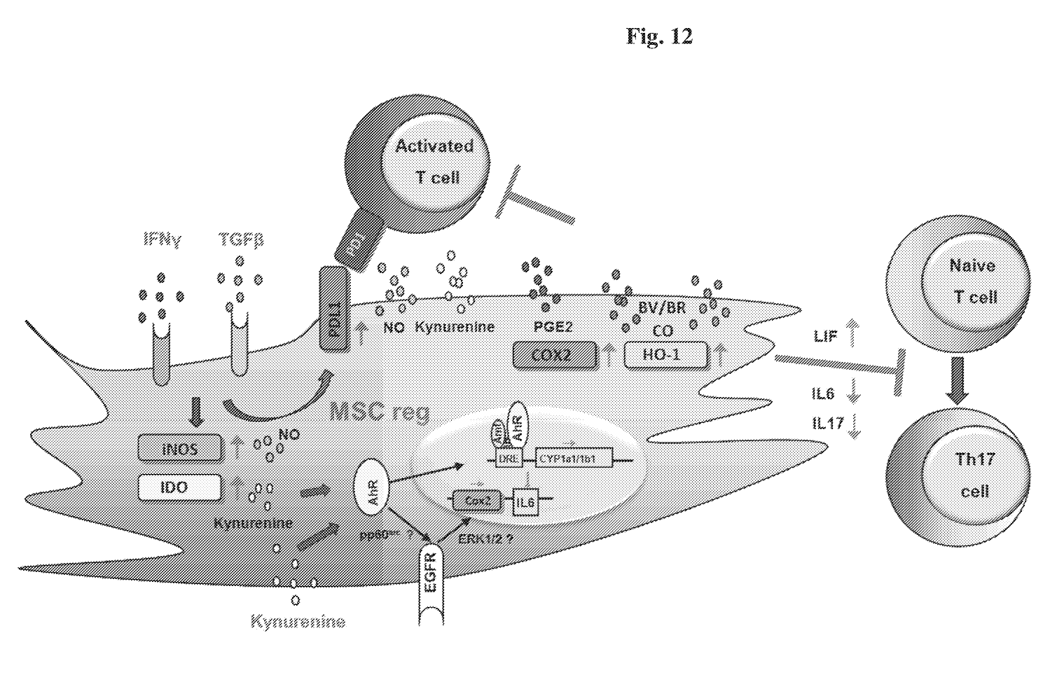

Mice were transplanted and injected with TCT cells as was done in the past (not shown). To evaluate the effects of TCT treatment on the hematological reconstitution after transplantation we collected blood samples from the tail vain of the mice at day 13 after transplantation and evaluated the number of hematopoietic cells by blood count (CBC). We found no effect on the number of white blood cells as compared to control mice (data not shown). However, the number of platelets was significantly higher in the TCT treated mice (FIG. 12A).

In our previous in vitro experiments, we found that TCT cells significantly reduced the secretion of IL17 from both murine and human activated lymphocytes in co-culture. This effect was associated with an elevated expression of the regulatory cytokine LIF in the TCT cells. IFNγ levels were reduced both by TCT and non-treated MSCs. In order to find out if the attenuating effect of TCT cells on GVHD development is associated with cytokine regulation, we collected blood plasma from the mice on days 13 and 20 after transplantation and evaluated the levels of different inflammatory and regulatory cytokines using Milliplex Magpix System.

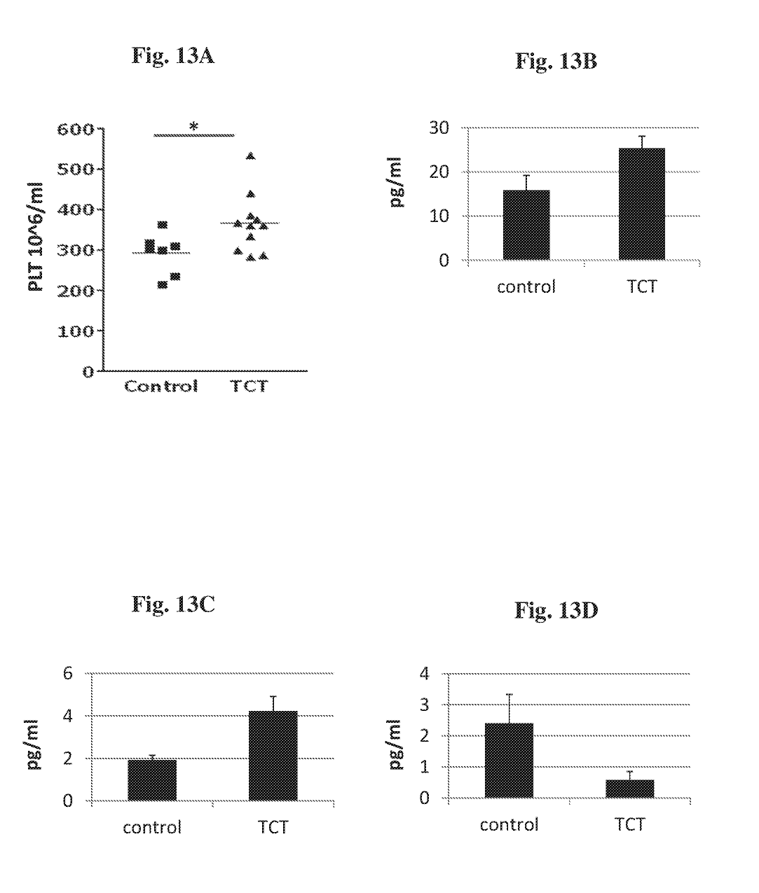

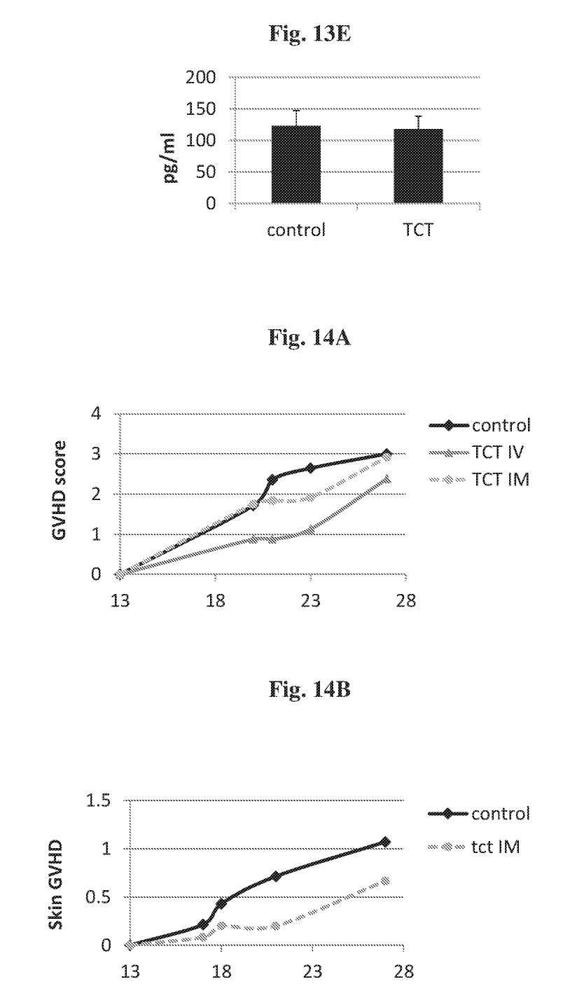

In accordance with our in vitro results, we observed an elevation in the plasma levels of LIF (FIG. 13C) and the regulatory cytokine IL10 (FIG. 13B) at day 13 after transplantation, before the development of any clinical signs of GVHD (FIG. 13B). This was followed by decreased levels of IL17 at day 20 (FIG. 13D). The levels of IFNγ were not affected (FIG. 13E).

### EXAMPLE 8

### Intravenous Versus Intramuscular Administration

Currently, MSC cell therapy is administered intravenous (IV) to GVHD patients. However, the literature has indicated a beneficial effect for intramuscular (IM) administration of MSCs. We therefor compared the two modes of administration (not shown). Our results show that IM administration could not reduce the total GVHD score, like we showed for IV administration. However, IM administration significantly reduced skin GVHD (FIGS. 14A and B).

### EXAMPLE 9

### Prolonged Effect of TCT Cell Therapy with an Additional Point of Administration

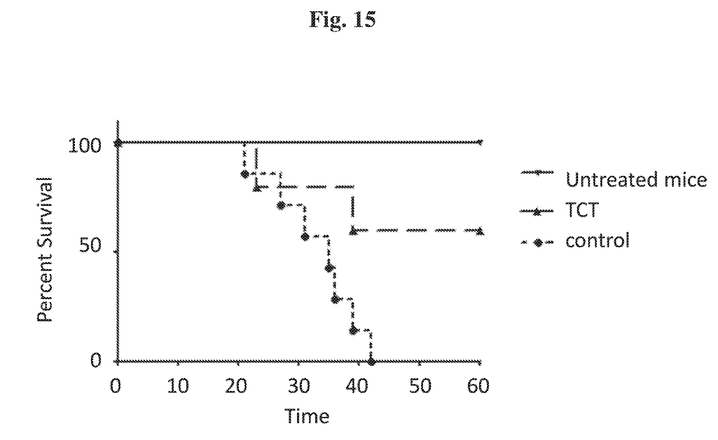

Since our previous results showed that single administration of TCT cells attenuates GVHD for an average of 25 days, we wanted to examine whether an additional administration after the onset of the disease could extend the regulatory effect. To this aim we added a second administration point at day 22 after transplantation. At the second administration, 0.1 million TCT cells were injected IM in the legs of the mice (not shown). This treatment significantly improved mice survival to more than 2 month after transplantation (FIG. 15).

### EXAMPLE 10

### TCT-Treated Primary Myeloid-Derived Suppressor Cell Lines

(i) Primary myeloid-derived suppressor cell are obtained from mice or humans according to methods well known in the art (Wu, Zhao et al. 2014).

(ii) The inhibitory effect of TCT-treated primary myeloid-derived suppressor cell lines on T cell proliferation and secretion of inflammatory cytokines is assessed using methods described above for MSCs.

(ii) The effect of TCT-treatment on the regulatory phenotype of human primary myeloid-derived suppressor cell lines is assessed by measuring expression levels of for example IDO and iNOS, PD-L1, MHC I, MHC II, COX2, IL6 etc.

(iii) The inhibition of acute GVHD and improvement of survival in semi-allogeneic BM transplantation mouse model is assessed. A semi-allogeneic GVHD mouse model is used as previously described (Azar, Shainer et al. 2013). Briefly, recipient F1 mice receive lethal whole-body irradiation and are reconstituted with 8*10⁶ donor C57BL/6 BM cells and 1*10⁷ spleen cells the following day. Concurrently, treated/untreated primary myeloid-derived suppressor cells are injected IV. For GVHD evaluation, mice are monitored daily for weight loss, diarrhea, ruffled skin, and survival (Samuel, Azar et al. 2008). GVHD score, based on all of the aforementioned factors (rated on a scale of 0-4), is calculated (Przepiorka, Weisdorf et al. 1995).

(iv) The effect of TCT-treatment on platelet recovery is investigated as described for MSCs above.

### REFERENCES

Aggarwal, S. and M. F. Pittenger (2005). “Human mesenchymal stem cells modulate allogeneic immune cell responses.” Blood 105(4): 1815-1822.

Aisemberg, J., C. Vercelli, S. Billi, M. L. Ribeiro, D. Ogando, R. Meiss, S. M. McCann, V. Rettori and A. M. Franchi (2007). “Nitric oxide mediates prostaglandins' deleterious effect on lipopolysaccharide-triggered murine fetal resorption.” Proc Natl Acad Sci U S A 104(18): 7534-7539.

Allison, M. (2009). “Genzyme backs Osiris, despite Prochymal flop.” Nat Biotechnol 2.966-967:(11)7

Ankrum, J. and J. M. Karp (2010). “Mesenchymal stem cell therapy: Two steps forward, one step back.” Trends Mol Med 16(5): 203-209.

Azar, Y., R. Shainer, O. Almogi-Hazan, R. Bringer, S. R. Compton, M. J. Paidas, E. R. Barnea and R. Or (2013) “Preimplantation factor reduces graft-versus-host disease by regulating immune response and lowering oxidative stress (murine model).” Biol Blood Marrow Transplant 19(4): 519-528.

Casiraghi, F., N. Perico and G. Remuzzi (2013). “Mesenchymal stromal cells to promote solid organ transplantation tolerance.” Curr Opin Organ Transplant 18(1): 51-58.

Chinnadurai, R., I. B. Copland, S. R. Patel and J. Galipeau (2014). “IDO-independent suppression of T cell effector function by IFN-gamma-licensed human mesenchymal stromal cells.” J Immunol 192(4): 1491-1501.

Chitteti, B. R., M. Bethel, M. A. Kacena and E. F. Srour (2013). “CD166 and regulation of hematopoiesis.” Curr Opin Hematol 20(4): 273-280.

Chitteti, B. R., M. Kobayashi, Y. Cheng, H. Zhang, B. A. Poteat, H. E. Broxmeyer, L. M. Pelus, H. Hanenberg, A. Zollman, M. M. Kamocka, N. Carlesso, A. A. Cardoso, M. A. Kacena and E. F. Srour (2013). “CD166 regulates human and murine hematopoietic stem cells and the hematopoietic niche.” Blood.

Cole, K. E., C. A. Strick, T. J. Paradis, K. T. Ogborne, M. Loetscher, R. P. Gladue, W. Lin, J. G. Boyd, B. Moser, D. E. Wood, B. G. Sahagan and K. Neote (1998). “Interferon-inducible T cell alpha chemoattractant (I-TAC): a novel non-ELR CXC chemokine with potent activity on activated T cells through selective high affinity binding to CXCR3.” J Exp Med 187(12): 2009-2021.

Dander, E., A. Balduzzi, G. Zappa, G. Lucchini, P. Perseghin, V. Andre, E. Todisco, D. Rahal, M. Migliavacca, D. Longoni, G. Solinas, A. Villa, E. Berti, P. D. Mina, M. Parma, P. Allavena, E. Biagi, A. Rovelli, A. Biondi and G. D'Amico (2009). “Interleukin-17-producing T-helper cells as new potential player mediating graft-versus-host disease in patients undergoing allogeneic stem-cell transplantation.” Transplantation 88(11): 1261-1272.

Duffy, M. M., J. Pindjakova, S. A. Hanley, C. McCarthy, G. A. Weidhofer, E. M. Sweeney, K. English, G. Shaw, J. M. Murphy, F. P. Barry, B. P. Mahon, O. Belton, R. Ceredig and M. D. Griffin (2012). “Mesenchymal stem cell inhibition of T-helper 17 cell-differentiation is triggered by cell-cell contact and mediated by prostaglandin E2 via the EP4 receptor.” Eur J Immunol 41(10): 2840-2851.

Dufour, J. H., M. Dziejman, M. T. Liu, J. H. Leung, T. E. Lane and A. D. Luster. (2002) “IFN-gamma-inducible protein 10 (IP-10; CXCL10)-deficient mice reveal a role for IP-10 in effector T cell generation and trafficking.” J Immunol 168(7): 3195-3204.

English, K., J. M. Ryan, L. Tobin, M. J. Murphy, F. P. Barry and B. P. Mahon (2009). “Cell contact, prostaglandin E(2) and transforming growth factor beta 1 play non-redundant roles in human mesenchymal stem cell induction of CD4+CD25(High) forkhead box P3+ regulatory T cells.” Clin Exp Immunol 156(1): 149-160.

Figueroa, F. E., F. Carrion, S. Villanueva and M. Khoury (2012). “Mesenchymal stem cell treatment for autoimmune diseases: a critical review.” Biol Res 45(3): 269-277.

Forbes, G. M., M. J. Sturm, R. W. Leong, M. P. Sparrow, D. Segarajasingam, A. G. Cummins, M. Phillips and R. P. Herrmann”. (2014) A phase 2 study of allogeneic mesenchymal stromal cells for luminal Crohn's disease refractory to biologic therapy.” Clin Gastroenterol Hepatol 12(1): 64-71.

Francois, M., R. Romieu-Mourez, M. Li and J. Galipeau (2011). “Human MSC Suppression Correlates With Cytokine Induction of Indoleamine 2,3-Dioxygenase and Bystander M2 Macrophage Differentiation.” Mol Ther.

Francois, M., R. Romieu-Mourez, S. Stock-Martineau, M. N. Boivin, J. L. Bramson and J. Galipeau (2009). “Mesenchymal stromal cells cross-present soluble exogenous antigens as part of their antigen-presenting cell properties.” Blood 114(13): 2632-2638.

Fritsche, E., C. Schafer, C. Calles, T. Bernsmann, T. Bernshausen, M. Wurm, U. Hubenthal, J. E. Cline, H. Hajimiragha, P. Schroeder, L. O. Klotz, A. Rannug, P. Furst, H. Hanenberg, J. Abel and J. Krutmann (2007). “Lightening up the UV response by identification of the arylhydrocarbon receptor as a cytoplasmatic target for ultraviolet B radiation.” Proc Natl Acad Sci U S A 104(21): 8851-8856.

Gao, W., L. Thompson, Q. Zhou, P. Putheti, T. M. Fahmy, T. B. Strom and S. M. Metcalfe (2009). “Treg versus Th17 lymphocyte lineages are cross-regulated by LIF versus IL-6.” Cell Cycle 8(9): 1444-1450.

Gavalas, N. G., M. Tsiatas, O. Tsitsilonis, E. Politi, K. Ioannou, A. C. Ziogas, A. Rodolakis, G. Vlahos, N. Thomakos, D. Haidopoulos, E. Terpos, A. Antsaklis, M. A. Dimopoulos and A. Bamias (2012). “VEGF directly suppresses activation of T cells from ascites secondary to ovarian cancer via VEGF receptor type 2.” Br J Cancer 107(11): 1869-1875.

Ge, W., J. Jiang, J. Arp, W. Liu, B. Garcia and H. Wang (2010). “Regulatory T-cell generation and kidney allograft tolerance induced by mesenchymal stem cells associated with indoleamine 2,3 -dioxygenase expression”. Transplantation 90(12): 1312-1320.

Ghannam, S., J. Pene, G. Torcy-Moquet, C. Jorgensen and H. Yssel (2010). “Mesenchymal Stem Cells Inhibit Human Th17 Cell Differentiation and Function and Induce a T Regulatory Cell Phenotype.” J Immunol.

Glenn, J. D., M. D. Smith, P. A. Calabresi and K. A. Whartenby (2014). “Mesenchymal Stem Cells Differentially Modulate Effector CD8+ T Cell Subsets and Exacerbate Experimental Autoimmune Encephalomyelitis.” Stem Cells 32(10): 2744-2755.

Glennie, S., I. Soeiro, P. J. Dyson, E. W. Lam and F. Dazzi (2005). “Bone marrow mesenchymal stem cells induce division arrest anergy of activated T cells.” Blood 105(7): 2821-2827.

Greenbaum, A., Y. M. Hsu, R. B. Day, L. G. Schuettpelz, M. J. Christopher, J. N. Borgerding, T. Nagasawa and D. C. Link (2013). “CXCL12 in early mesenchymal progenitors is required for haematopoietic stem-cell maintenance.” Nature 495(7440): 227-230.

Hanieh, H. (2014). “Toward Understanding the Role of Aryl Hydrocarbon Receptor in the Immune System: Current Progress and Future Trends.” Biomed Res Int 2014: 520763.

Jasperson, L. K., C. Bucher, A. Panoskaltsis-Mortari, A. L. Mellor, D. H. Munn and B. R. Blazar (2009). “Inducing the tryptophan catabolic pathway, indoleamine 2,3-dioxygenase (IDO), for suppression of graft-versus-host disease (GVHD) lethality.” Blood 3; 114(24):5062-70.

Kappel, L. W., G. L. Goldberg, C. G. King, D. Y. Suh, O. M. Smith, C. Ligh, A. M. Holland, J. Grubin, N. M. Mark, C. Liu, Y. Iwakura, G. Heller and M. R. van den Brink (2009). “IL-17 contributes to CD4-mediated graft-versus-host disease.” Blood 113(4): 945-952.

Kim, N., K. I. Im, J. Y. Lim, E. J. Jeon, Y. S. Nam, E. J. Kim and S. G. Cho (2013). “Mesenchymal stem cells for the treatment and prevention of graft-versus-host disease: experiments and practice.” Ann Hematol 92(10): 1295-1308.

Kim, S. F. (2011). “The role of nitric oxide in prostaglandin biology; update.” Nitric Oxide 25(3): 255-264.

Kimura, A., T. Naka, T. Nakahama, I. Chinen, K. Masuda, K. Nohara, Y. Fujii-Kuriyama and T. Kishimoto”. (2009) Aryl hydrocarbon receptor in combination with Stat1 regulates LPS-induced inflammatory responses.” J Exp Med 206(9): 2027-2035.

Kobayashi, Y. (2008). “The role of chemokines in neutrophil biology.”Front Biosci 13: 2400-2407.

Le Blanc, K. and D. Mougiakakos (2012). “Multipotent mesenchymal stromal cells and the innate immune system.” Nat Rev Immunol 12(5): 383-396.

Le Blanc, K., C. Tammik, K. Rosendahl, E. Zetterberg and O. Ringden (2003). “HLA expression and immunologic properties of differentiated and undifferentiated mesenchymal stem cells.” Exp Hematol 31(10): 890-896.

Li, H., Z. Guo, X. Jiang, H. Zhu, X. Li and N. Mao (2008). “Mesenchymal stem cells alter migratory property of T and dendritic cells to delay the development of murine lethal acute graft-versus-host disease.” Stem Cells 26(10): 2531-2541.

Li, W., G. Ren, Y. Huang, J. Su, Y. Han, J. Li, X. Chen, K. Cao, Q. Chen, P. Shou, L. Zhang, Z. R. Yuan, A. I. Roberts, S. Shi, A. D. Le and Y. Shi (2012). “Mesenchymal stem cells: a double-edged sword in regulating immune responses.” Cell Death Differ.

Liang, J., H. Zhang, B. Hua, H. Wang, L. Lu, S. Shi, Y. Hou, X. Zeng, G. S. Gilkeson and L. Sun (2010). “Allogenic mesenchymal stem cells transplantation in refractory systemic lupus erythematosus: a pilot clinical study.” Ann Rheum Dis 69(8): 1423-1429.

Luz-Crawford, P., D. Noel, X. Fernandez, M. Khoury, F. Figueroa, F. Carrion, C. Jorgensen and F. Djouad (2012). “Mesenchymal stem cells repress Th17 molecular program through the PD-1 pathway.” PLoS One 7(9): e45272.

Meisel, R., S. Brockers, K. Heseler, O. Degistirici, H. Bulle, C. Woite, S. Stuhlsatz, W. Schwippert, M. Jager, R. Sorg, R. Henschler, J. Seissler, D. Dilloo and W. Daubener (2011). “Human but not murine multipotent mesenchymal stromal cells exhibit broad-spectrum antimicrobial effector function mediated by indoleamine 2,3-dioxygenase.” Leukemia 25(4): 648-654.

Mellor, A. L. and D. H. Munn (2004). “IDO expression by dendritic cells: tolerance and tryptophan catabolism.” Nat Rev Immunol 4.762-774 :(10)

Mezrich, J. D., J. H. Fechner, X. Zhang, B. P. Johnson, W. J. Burlingham and C. A. Bradfield (2010). “An interaction between kynurenine and the aryl hydrocarbon receptor can generate regulatory T cells.” J Immunol 185(6): 3190-3198.

Newell, L. F., R. J. Deans and R. T. Maziarz (2014). “Adult adherent stromal cells in the management of graft-versus-host disease.” Expert Opin Biol Ther 14(2): 231-246.

Nguyen, N. T., A. Kimura, T. Nakahama, I. Chinen, K. Masuda, K. Nohara, Y. Fujii-Kuriyama and T. Kishimoto (2010). “Aryl hydrocarbon receptor negatively regulates dendritic cell immunogenicity via a kynurenine-dependent mechanism.” Proc Natl Acad Sci U S A 107(46): 19961-19966.

Omatsu, Y., M. Seike, T. Sugiyama, T. Kume and T. Nagasawa”. (2014) Foxc1 is a critical regulator of haematopoietic stem/progenitor cell niche formation.” Nature.

Park, J., W. Gao, R. Whiston, T. B. Strom, S. Metcalfe and T. M. Fahmy (2011). “Modulation of CD4+ T lymphocyte lineage outcomes with targeted, nanoparticle-mediated cytokine delivery.” Mol Pharm 8(1): 143-152.

Plumas, J., L. Chaperot, M. J. Richard, J. P. Molens, J. C. Bensa and M. C. Favrot (2005). “Mesenchymal stem cells induce apoptosis of activated T cells.” Leukemia 19(9): 1597-1604.

Przepiorka, D., D. Weisdorf, P. Martin, H. G. Klingemann, P. Beatty, J. Hows and E. D. Thomas (1995). “1994 Consensus Conference on Acute GVHD Grading.” Bone Marrow Transplant 15(6): 825-828.

Rasmusson, I., O. Ringden, B. Sundberg and K. Le Blanc (2005). “Mesenchymal stem cells inhibit lymphocyte proliferation by mitogens and alloantigens by different mechanisms.” Exp Cell Res 305(1): 33-41.

Rasmusson, I., M. Uhlin, K. Le Blanc and V. Levitsky (2007). “Mesenchymal stem cells fail to trigger effector functions of cytotoxic T lymphocytes.” J Leukoc Biol 82(4): 887-893.

Ratajczak, P., A. Janin, R. Peffault de Latour, C. Leboeuf, A. Desveaux, K. Keyvanfar, M. Robin, E. Clave, C. Douay, A. Quinquenel, C. Pichereau, P. Bertheau, J. Y. Mary and G. Socie (2010). “Th17/Treg ratio in human graft-versus-host disease.” Blood 116(7): 1165-1171.

Ren, G., J. Su, L. Zhang, X. Zhao, W. Ling, A. L'Huillie, J. Zhang, Y. Lu, A. I. Roberts, W. Ji, A. B. Rabson and Y. Shi (2009). “Species Variation in the Mechanisms of Mesenchymal Stem Cell-Mediated Immunosuppression.” Stem Cells.

Ren, G., L. Zhang, X. Zhao, G. Xu, Y. Zhang, A. I. Roberts, R. C. Zhao and Y. Shi (2008). “Mesenchymal stem cell-mediated immunosuppression occurs via concerted action of chemokines and nitric oxide.” Cell Stem Cell 2. 141-150:(2)

Resnick, I. B., C. Barkats, M. Y. Shapira, P. Stepensky, A. I. Bloom, A. Shimoni, D. Mankuta, N. Varda-Bloom, L. Rheingold, M. Yeshurun, B. Bielorai, A. Toren, T. Zuckerman, A. Nagler and R. Or (2013). “Treatment of severe steroid resistant acute GVHD with mesenchymal stromal cells (MSC).” Am J Blood Res 3(3): 225-238.

Romieu-Mourez, R., M. Francois, M. N. Boivin, M. Bouchentouf, D. E. Spaner and J. Galipeau (2009). “Cytokine modulation of TLR expression and activation in mesenchymal stromal cells leads to a proinflammatory phenotype.” J Immunol 182(12): 7963-7973.

Romieu-Mourez, R., M. Francois, M. N. Boivin, J. Stagg and J. Galipeau (2007). “Regulation of MHC class II expression and antigen processing in murine and human mesenchymal stromal cells by IFN-gamma, TGF-beta, and cell density.” J Immunol 179(3): 1549-1558.

Ryan, J. M., F. Barry, J. M. Murphy and B. P. Mahon (2007). “Interferon-gamma does not break, but promotes the immunosuppressive capacity of adult human mesenchymal stem cells.” Clin Exp Immunol 149(2): 353-363.

Samuel, S., Y. Azar, N. Corchia and R. Or (2008). “Improved immune function with donor B-cell infusion after semi-allogeneic bone marrow transplantation in mice.” Arch Med Res 39(1): 61-68.

Sato, K., K. Ozaki, I. Oh, A. Meguro, K. Hatanaka, T. Nagai, K. Muroi and K. Ozawa (2007). “Nitric oxide plays a critical role in suppression of T-cell proliferation by mesenchymal stem cells.” Blood 109(1): 228-234.

Sheng, H., Y. Wang, Y. Jin, Q. Zhang, Y. Zhang, L. Wang, B. Shen, S. Yin, W. Liu, L. Cui and N. Li (2008). “A critical role of IFNgamma in priming MSC-mediated suppression of T cell proliferation through up-regulation of B7-H1.” Cell Res 18(8): 846-857.

Soria, G. and A. Ben-Baruch (2008). “The inflammatory chemokines CCL2 and CCL5 in breast cancer.” Cancer Lett 267(2): 271-285.

Stagg, J., S. Pommey, N. Eliopoulos and J. Galipeau (2006). “Interferon-gamma-stimulated marrow stromal cells: a new type of nonhematopoietic antigen-presenting cell.” Blood 107(6): 2570-2577.

Sundin, M., A. J. Barrett, O. Ringden, M. Uzunel, H. Lonnies, A. L. Dackland, B. Christensson and K. L. Blanc (2009). “HSCT recipients have specific tolerance to MSC but not to the MSC donor.” J Immunother 32(7): 755-764.

Svobodova, E., M. Krulova, A. Zajicova, K. Pokorna, J. Prochazkova, P. Trosan and V. Holan (2011). “The Role of Mouse Mesenchymal Stem Cells in Differentiation of Naive T-Cells into Anti-Inflammatory Regulatory T-Cell or Proinflammatory Helper T-Cell 17 Population.” Stem Cells Dev.

Vogel, C. F,. S. R. Goth, B. Dong, I. N. Pessah and F. Matsumura (2008). “Aryl hydrocarbon receptor signaling mediates expression of indoleamine 2,3-dioxygenase.” Biochem Biophys Res Commun 375(3): 331-335.

Wang, S., X. Qu and R. C. Zhao (2012). “Clinical applications of mesenchymal stem cells.” J Hematol Oncol 5: 19.

Weinberg, J. B. (2000). “Nitric oxide synthase 2 and cyclooxygenase 2 interactions in inflammation.” Immunol Res 22(2-3): 319-341.

Wu, T, Y Zhao and Y Zhao (2014) “The roles of myeloid-derived suppressor cells in transplantation.” Expert Rev Clin Immunol. 2014 October; 10(10):1385-94.

Yamout, B., R. Hourani, H. Salti, W. Barada, T. El-Hajj, A. Al-Kutoubi, A. Herlopian, E. K. Baz, R. Mahfouz, R. Khalil-Hamdan, N. M. Kreidieh, M. El-Sabban and A. Bazarbachi (2010). “Bone marrow mesenchymal stem cell transplantation in patients with multiple sclerosis: a pilot study.” J Neuroimmunol 227(1-2): 185-189.

1-34. (canceled)

35. A method for inhibiting proliferation of T cells comprising administering to subject in need thereof
(i) a regulatory mesenchymal stem cell line or regulatory myeloid-derived suppressor cell line obtained by a method comprising contacting ex-vivo a primary mesenchymal stem cell line or a primary myeloid-derived suppressor cell line with (a) TGF-β as a sole active agent prior to contacting said cell line with (b) a combination of TGFβ, an inflammatory agent and a tryptophan IDO metabolite, wherein optionally said administering comprises administration of said regulatory mesenchymal stem cell line or regulatory myeloid-derived suppressor cell line on at least two occasions; or
(ii) a regulatory mesenchymal stem cell line or regulatory myeloid-derived suppressor cell line obtained by a method comprising contacting ex-vivo a primary mesenchymal stem cell line or a primary myeloid-derived suppressor cell line with a combination of TGFβ, an inflammatory agent and a tryptophan IDO metabolite, thereby obtaining said regulatory myeloid-derived suppressor cell line, wherein said administering comprises administration of said regulatory mesenchymal stem cell line or regulatory myeloid-derived suppressor cell line on at least two occasions.

36. The method according to claim 35, wherein said inflammatory agent is selected from IFNγ, TNFα, IL-1 or LPS and said tryptophan IDO metabolite is independently selected from kynurenine, N-formylkynurenine, 3-hydroxykynurenine, 3-hydroxyanthranilic acid or quinolinate.

37. The method according to claim 36, wherein said inflammatory agent is IFNγ.

38. The method according to claim 36, wherein said tryptophan IDO metabolite is kynurenine.

39. A method for reducing Th17 or Tc17 differentiation of activated T cells in an individual comprising administering to subject in need thereof a regulatory mesenchymal stem cell line or regulatory myeloid-derived suppressor cell line obtained by a method comprising contacting ex-vivo a primary mesenchymal stem cell line or a primary myeloid-derived suppressor cell line with a combination of TGFβ, an inflammatory agent and a tryptophan IDO metabolite, thereby obtaining said regulatory myeloid-derived suppressor cell line, wherein said cell line is optionally contacted with TGFβ as a sole active agent prior to contacting said cell line with said combination of TGFβ, an inflammatory agent and a tryptophan IDO metabolite.

40. The method according to claim 39, wherein said administering comprises administration of said regulatory mesenchymal stem cell line or regulatory myeloid-derived suppressor cell line on at least two occasions.

41. The method according to claim 39, wherein said inflammatory agent is selected from IFNγ, TNFα, IL-1 or LPS and said tryptophan IDO metabolite is independently selected from kynurenine, N-formylkynurenine, 3-hydroxykynurenine, 3-hydroxyanthranilic acid or quinolinate.

42. The method according to claim 41, wherein said inflammatory agent is IFNγ.

43. The method according to claim 41, wherein said tryptophan IDO metabolite is kynurenine.

44. A method for reducing an inflammatory response comprising administering to subject in need thereof
(i) a regulatory mesenchymal stem cell line or regulatory myeloid-derived suppressor obtained by a method comprising contacting ex-vivo a primary mesenchymal stem cell line or a primary myeloid-derived suppressor cell line with (a) TGF-β as a sole active agent prior to contacting said cell line with (b) a combination of TGFβ, an inflammatory agent and a tryptophan IDO metabolite, wherein optionally said administering comprises administration of said regulatory mesenchymal stem cell line or regulatory myeloid-derived suppressor cell line on at least two occasions; or
(ii) a regulatory mesenchymal stem cell line or regulatory myeloid-derived suppressor cell line obtained by a method comprising contacting ex-vivo a primary mesenchymal stem cell line or a primary myeloid-derived suppressor cell line with a combination of TGFβ, an inflammatory agent and a tryptophan IDO metabolite, thereby obtaining said regulatory myeloid-derived suppressor cell line, wherein said administering comprises administration of said regulatory mesenchymal stem cell line or regulatory myeloid-derived suppressor cell line on at least two occasions.

45. The method according to claim 44, wherein said inflammatory agent is selected from IFNγ, TNFα, IL-1 or LPS and said tryptophan IDO metabolite is independently selected from kynurenine, N-formylkynurenine, 3-hydroxykynurenine, 3-hydroxyanthranilic acid or quinolinate.

46. The method according to claim 45, wherein said inflammatory agent is IFNγ.

47. The method according to claim 45, wherein said tryptophan IDO metabolite is kynurenine.

48. A method for treating or preventing graft-versus-host disease (GVHD), transplanted organ rejection, an autoimmune disease, an inflammatory disease, allergy or an immune-mediated neurodegenerative disorder comprising administering to subject in need thereof
(i) a regulatory mesenchymal stem cell line or regulatory myeloid-derived suppressor cell line obtained by a method comprising contacting ex-vivo a primary mesenchymal stem cell line or a primary myeloid-derived suppressor cell line with (a) TGF-β as a sole active agent prior to contacting said cell line with (b) a combination of TGFβ, an inflammatory agent and a tryptophan IDO metabolite, wherein optionally said treating comprises administration of said regulatory mesenchymal stem cell line or regulatory myeloid-derived suppressor cell line on at least two occasions; or
(ii) a regulatory mesenchymal stem cell line or regulatory myeloid-derived suppressor cell line obtained by a method comprising contacting ex-vivo a primary mesenchymal stem cell line or a primary myeloid-derived suppressor cell line with a combination of TGFβ, an inflammatory agent and a tryptophan IDO metabolite, thereby obtaining said regulatory myeloid-derived suppressor cell line, wherein said administering comprises administration of said regulatory mesenchymal stem cell line or regulatory myeloid-derived suppressor cell line on at least two occasions.

49. The method according to claim 48, wherein said inflammatory agent is selected from IFNγ, TNFα, IL-1 or LPS and said tryptophan IDO metabolite is independently selected from kynurenine, N-formylkynurenine, 3-hydroxykynurenine, 3-hydroxyanthranilic acid or quinolinate.

50. The method according to claim 49, wherein said inflammatory agent is IFNγ.

51. The method according to claim 49, wherein said tryptophan IDO metabolite is kynurenine.

52. The method according to claim 48, wherein said treating results in a reduced total GVHD score.

53. The method according to claim 52, wherein said regulatory mesenchymal stem cell line or regulatory myeloid-derived suppressor cell line is administered by intravenous injection.

54. The method according to claim 48, wherein said treating results in a reduced skin GVHD score.

55. The method according claim 54, wherein said regulatory mesenchymal stem cell line or regulatory myeloid-derived suppressor cell line is administered by intramuscular injection.

56. A method for improving platelet recovery following organ transplantation comprising administering to subject in need thereof a regulatory mesenchymal stem cell line or regulatory myeloid-derived suppressor cell line obtained by a method comprising contacting ex-vivo a primary mesenchymal stem cell line or a primary myeloid-derived suppressor cell line with a combination of TGFβ, an inflammatory agent and a tryptophan IDO metabolite, thereby obtaining said regulatory myeloid-derived suppressor cell line, wherein said cell line is optionally contacted with TGFβ as a sole active agent prior to contacting said cell line with said combination of TGFβ, an inflammatory agent and a tryptophan IDO metabolite.

57. The method according to claim 56, wherein said administering comprises administration of said regulatory mesenchymal stem cell line or regulatory myeloid-derived suppressor cell line on at least two separate occasions.

58. The method according to claim 56, wherein said inflammatory agent is selected from IFNγ, TNFα, IL-1 or LPS and said tryptophan IDO metabolite is independently selected from kynurenine, N-formylkynurenine, 3-hydroxykynurenine, 3-hydroxyanthranilic acid or quinolinate.

59. The method according to claim 58, wherein said inflammatory agent is IFNγ.

60. The method according to claim 58, wherein said tryptophan IDO metabolite is kynurenine.
